(19) United States
(12) Patent Application Publication
(10) Pub. No.: US 2025/0017618 A1
(43) Pub. Date: Jan. 16, 2025

(54) MECHANICAL THROMBECTOMY ASSEMBLY WITH EMBOLIC PROTECTION, AND ASSOCIATED DEVICES, SYSTEMS, AND METHODS

(71) Applicant: Inari Medical, Inc., Irvine, CA (US)

(72) Inventors: Jakub Marek Truty, San Clemente, CA (US); Garrett Thomas Offerman, Orange, CA (US); Aaron Dederich, San Clemente, CA (US); Brian Edward Merritt, San Clemente, CA (US); Melissa Montejano Viveros, Lake Forest, CA (US); Alejandra Turcios, Newport Breach, CA (US); Brian Michael Strauss, San Clemente, CA (US)

(21) Appl. No.: 18/349,674

(22) Filed: Jul. 10, 2023

(51) Int. Cl. A61B 17/3207

(52) U.S. Cl. CPC A61B 17/320725; A61B 2017/22067; A61B 2017/22079

(57) ABSTRACT

Disclosed herein are clot treatment systems including integrated/systemized mechanical thrombectomy devices and clot treatment devices, and associated devices and methods. In some embodiments, a method of removing clot material from a blood vessel of patient includes inserting a catheter to proximate the clot material within the blood vessel while radially constraining a clot treatment device and an embolic protection device within the catheter. The method can further include moving the catheter proximally to deploy the clot treatment device at least partially distal to clot material within the blood vessel, and then moving the catheter further proximally to deploy the embolic protection device at least partially proximal to the clot material.

### TECHNICAL FIELD

The present technology generally relates to mechanical thrombectomy assemblies, including mechanical thrombectomy assemblies with embolic protection, and associated devices, systems, and methods.

### BACKGROUND

Thromboembolic events are characterized by an occlusion of a blood vessel. Thromboembolic disorders, such as stroke, pulmonary embolism, heart attack, peripheral thrombosis, atherosclerosis, and the like, affect many people. These disorders are a major cause of morbidity and mortality.

When an artery is occluded by a clot, tissue ischemia develops. The ischemia will progress to tissue infarction if the occlusion persists. However, infarction does not develop or is greatly limited if the flow of blood is reestablished rapidly. Failure to reestablish blood flow can accordingly lead to the loss of limb, angina pectoris, myocardial infarction, stroke, or even death.

In the venous circulation, occlusive material can also cause serious harm. Blood clots can develop in the large veins of the legs and pelvis, a common condition known as deep venous thrombosis (DVT). DVT commonly occurs where there is a propensity for stagnated blood (e.g., long-distance air travel, immobility, etc.) and clotting (e.g., cancer; recent surgery, such as orthopedic surgery, etc.). DVT can obstruct drainage of venous blood from the legs, leading to swelling, ulcers, pain, and infection. DVT can also create a reservoir in which blood clots can collect and then travel to other parts of the body, including the heart, lungs, brain (which may cause a stroke), abdominal organs, and/or extremities.

In the arterial circulation (e.g., the peripheral arterial circulation, the pulmonary circulation), the undesirable material can cause harm by obstructing different arteries. For example, an obstruction within the pulmonary arteries is a condition known as pulmonary embolism. If the obstruction is upstream, in the main or large branch pulmonary arteries, it can severely compromise total blood flow within the lungs, and therefore the entire body, and result in low blood pressure and shock. If the obstruction is downstream, in large to medium pulmonary artery branches, it can prevent a significant portion of the lung from participating in the exchange of gases to the blood resulting in low blood oxygen and buildup of blood carbon dioxide.

Various devices exist for performing a thrombectomy or removing clot material to reestablish blood flow within a patient. For example, mechanical thrombectomy catheters are a type of catheter used for thrombectomy, embolectomy, or retrieval procedures and often include an element on the distal end which serves to capture thrombi, emboli, foreign matter, and/or other particulates. The element may be made from wire, laser cut metal including nitinol, looped or braided wire, or an inflated element such as a balloon. However, the element on the distal end of the mechanical thrombectomy catheter can cause embolization of the clot material as the element is inserted and/or retracted through the clot material. The embolized clot material can flow to other parts of the body, which may lead to other medical complications.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1A is a partially schematic side view of a clot treatment system configured in accordance with embodiments of the present technology.

FIG. 1B is a side view of a clot treatment system configured in accordance with additional embodiments of the present technology.

FIG. 1C is a side view of a mechanical thrombectomy assembly of a clot treatment system configured in accordance with additional embodiments of the present technology.

FIGS. 2A and 2B are a side view and a perspective view, respectively, of a distal portion of the clot treatment system of FIG. 1A with a different clot treatment device configured in accordance with embodiments of the present technology.

FIGS. 3 and 4 are side views of respective clot removal elements of a clot treatment system configured in accordance with additional embodiments of the present technology.

FIG. 5 is a perspective view of a mechanical thrombectomy assembly configured in accordance with additional embodiments of the present technology.

FIG. 6 is a flow diagram of a process or method for removing clot material from a blood vessel of a patient using a clot treatment system in accordance with embodiments of the present technology.

FIGS. 7A-7E are side views of a distal portion of a clot treatment system during different stages of the process or method of FIG. 6 in accordance with embodiments of the present technology.

FIGS. 8A and 8B are a side view and a perspective view, respectively, of a distal portion of the clot treatment system of FIGS. 1B and 1C configured in accordance with additional embodiments of the present technology.

FIG. 9 is a perspective view of a distal portion of a clot treatment system configured in accordance with additional embodiments of the present technology.

FIG. 10 is a flow diagram of a process or method for removing clot material from a blood vessel of a patient using a clot treatment system in accordance with embodiments of the present technology.

FIGS. 11A-11E are side views of a distal portion of a clot treatment system during different stages of the process or method of FIG. 10 in accordance with embodiments of the present technology.

FIG. 12 is a flow diagram of a process or method for removing clot material from the vasculature of a patient using a clot treatment system in accordance with embodiments of the present technology.

FIGS. 13A-13G are side views of a clot treatment system during different stages of the process or method of FIG. 12 in accordance with embodiments of the present technology.

### DETAILED DESCRIPTION

The present technology is generally directed to clot treatment systems with integrated/systemized mechanical thrombectomy devices and clot treatment devices, and associated devices and methods. In some embodiments, an integrated clot treatment system includes an embolic protection device, a clot treatment device, a first shaft, and a second shaft. The embolic protection device can be carried by the first shaft and the clot treatment device can be carried by the second shaft. In some embodiments, the second shaft is slidably positioned within the first shaft. During a clot removal procedure, the clot treatment device and the embolic protection device can be inserted together while radially constrained within a delivery catheter into a blood vessel of patient including clot material to be treated. The delivery catheter can be retracted proximally to deploy the clot treatment device at least partially distal to clot material within the blood vessel, and then the delivery catheter can be further retracted proximally to deploy the embolic protection device at least partially proximal to the clot material. The clot treatment device can be used to mechanically engage and disrupt the clot material by, for example, retracting the clot treatment device proximally through the clot material and into the embolic protection device and/or the first shaft. The embolic protection device can be positioned to capture all or a portion of the clot material that embolizes or otherwise breaks off during mechanical engagement of the clot treatment device with the clot material and/or to direct clot material into the first shaft.

In some aspects of the present technology, the embolic protection device is integrated with the clot treatment device within the clot treatment system such that the clot treatment system can be easier to operate compared to other devices with separate embolic protection devices. In additional aspects of the present technology, the embolic protection device can be used to directly deploy the clot treatment device, which is expected to allow the clot treatment systems of the present technology to have a reduced profile (e.g., a smaller outer diameter) and/or to be easier to navigate through relatively smaller blood vessels, stenotic blockages, and/or thrombotic occlusions.

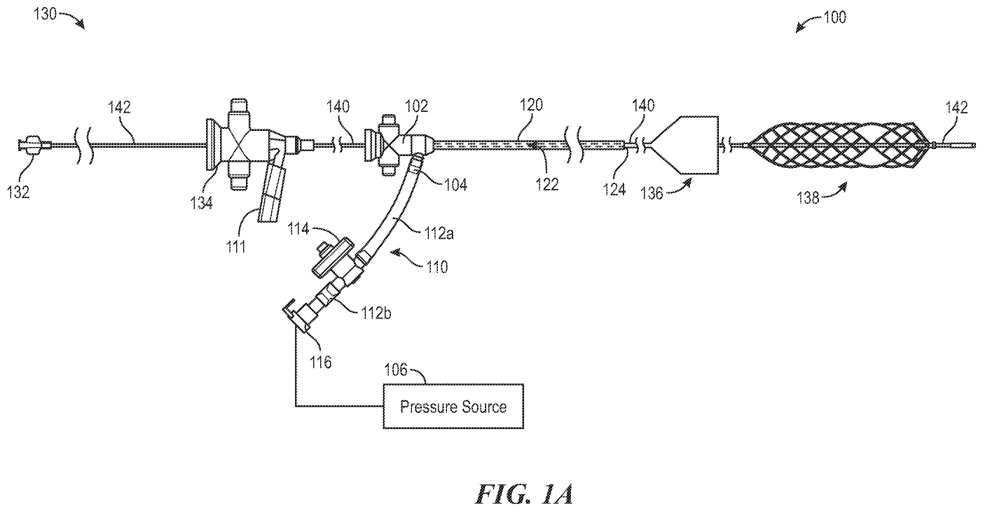

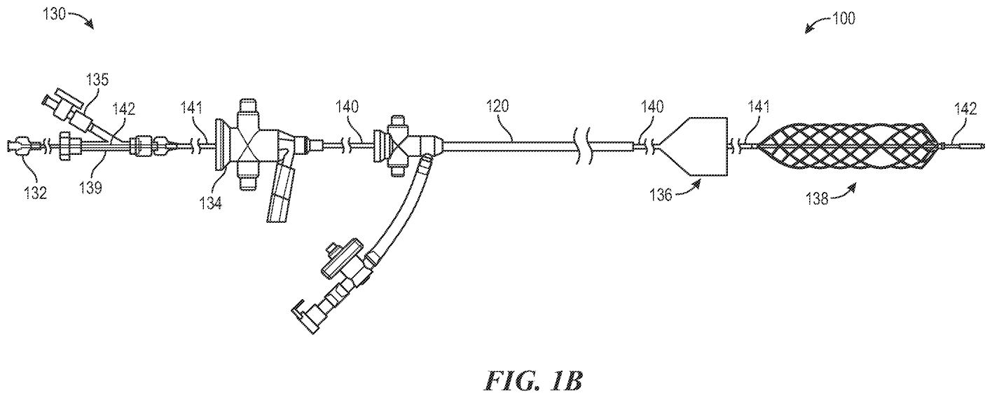

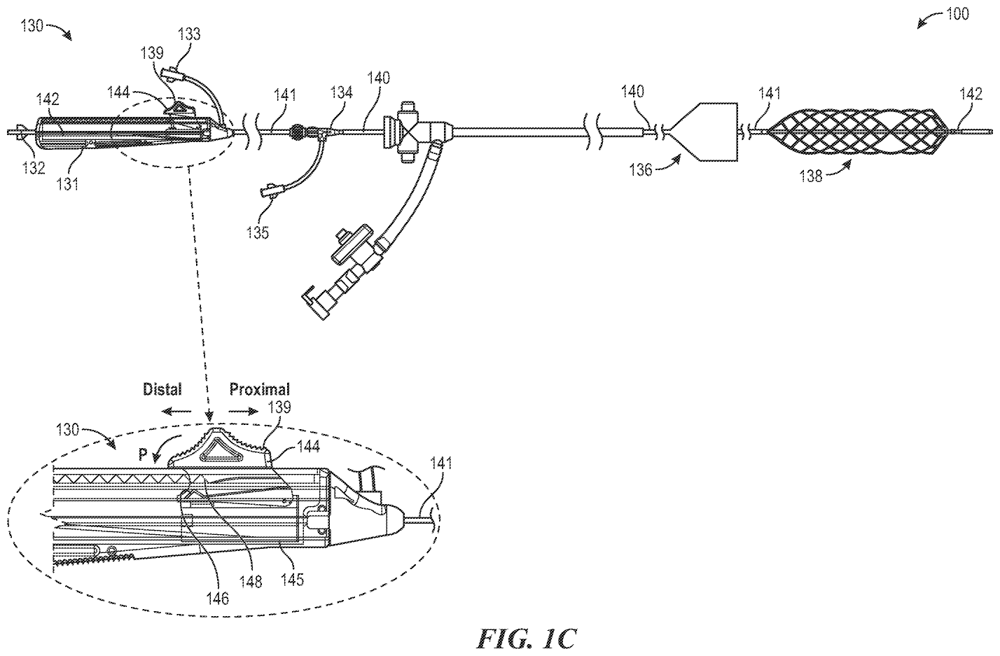

Certain details are set forth in the following description and in FIGS. 1A-13G to provide a thorough understanding of various embodiments of the present technology. In other instances, well-known structures, materials, operations, and/or systems often associated with intravascular procedures, clot removal procedures, clot treatment systems, clot treatment devices, embolic protection devices, catheters, and/or the like are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth. Moreover, although many of the devices and systems are described herein in the context of removing and/or treating clot material, the present technology can be used to remove and/or treat other unwanted material in addition or alternatively to clot material, such as thrombi, emboli, plaque, intimal hyperplasia, post-thrombotic scar tissue, etc. Accordingly, the terms “clot” and “clot material” as used herein can refer to any of the foregoing materials and/or the like.

The terminology used below is to be interpreted in its broadest reasonable manner, even though it is being used in conjunction with a detailed description of certain examples of embodiments of the technology. Indeed, certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles and other features shown in the Figures are merely illustrative of particular embodiments of the disclosure. Accordingly, other embodiments can have other details, dimensions, angles and features without departing from the present technology. In addition, those of ordinary skill in the art will appreciate that further embodiments of the present technology can be practiced without several of the details described below.

With regard to the terms “distal” and “proximal” within this description, unless otherwise specified, the terms can reference a relative position of the portions of a catheter subsystem with reference to an operator and/or a location in the vasculature. Also, as used herein, the designations “rearward.” “forward,” “upward,” “downward,” and the like are not meant to limit the referenced component to a specific orientation. It will be appreciated that such designations refer to the orientation of the referenced component as illustrated in the Figures; the systems of the present technology can be used in any orientation suitable to the user.

In the Figures, identical reference numbers identify identical, or at least generally similar, elements. To facilitate the discussion of any particular element, the most significant digit or digits of any reference number refers to the Figure in which that element is first introduced. For example, tubing assembly 110 is first introduced and discussed with reference to FIG. 1A.

To the extent any materials incorporated herein by reference conflict with the present disclosure, the present disclosure controls.

FIG. 1A is a partially schematic side view of a clot treatment system 100 (“the system 100”) configured in accordance with embodiments of the present technology. The system 100 can also be referred to as an aspiration assembly, a vascular access system, a clot removal system, a thrombectomy system, and/or the like. In the illustrated embodiment, the system 100 includes a tubing assembly 110 fluidly coupled to a catheter 120 via a valve 102. In some embodiments, the catheter 120 is an elongate member (e.g., a sheath, a shaft) configured to be inserted into and through a patient's vasculature and used to, for example, remove or otherwise treat clot material therein. In other embodiments, the catheter 120 can be an introducer sheath configured to be inserted through the skin and tissue tract of the patient to provide an access site through which other components (e.g., other catheters used to treat clot material) can traverse to easily access the vasculature. Accordingly, while referred to as “catheter 120,” the catheter 120 can comprise an introducer sheath, an access sheath, and/or another type of elongate member configured to be inserted through the skin and tissue tract and/or to traverse the vasculature of a patient. In general, the system 100 (i) can include features generally similar in structure and/or function, or identical in structure and/or function, to those of the clot treatment systems described in detail in U.S. patent application Ser. No. 16/536,185, filed Aug. 8, 2019, and titled “SYSTEM FOR TREATING EMBOLISM AND ASSOCIATED DEVICES AND METHODS,” which is incorporated herein by reference in its entirety, and/or (ii) can be used to treat/remove clot material from a patient (e.g., a human patient) using any of the methods described in detail therein.

The catheter 120 further defines a lumen 122 (shown in dashed line in FIG. 1A) extending entirely therethrough, e.g., from the valve 102 to a distal terminus 124 of the catheter 120. The catheter 120 can have varying lengths, flexibilities, shapes, thicknesses, and/or other properties in/along the various regions 122a-d. For example, the catheter 120 can comprise one or more coils, braids, and/or other structures positioned between one or more liner layers (e.g., inner and outer liner layers). In some embodiments, the catheter 120 can include several features generally similar or identical in structure and/or function to any of the catheters described in U.S. Patent Application No. 17/529,018, titled “CATHETERS HAVING SHAPED DISTAL PORTIONS, AND ASSOCIATED SYSTEMS AND METHODS,” and filed Nov. 17, 2021, U.S. patent application Ser. No. 17/529,064, titled “CATHETERS HAVING STEERABLE DISTAL PORTIONS, AND ASSOCIATED SYSTEMS AND METHODS,” and filed Nov. 17, 2021, and/or U.S. patent application Ser. No. 18/159,507, titled “ASPIRATION CATHETERS HAVING GROOVED INNER SURFACE, AND ASSOCIATE SYSTEM AND METHODS,” and filed Jan. 25, 2023, each of which is incorporated by reference herein in its entirety.

The valve 102 is fluidly coupled to the lumen 122 of the catheter 120 and can be integral with or coupled to the catheter 120 such that these components move together. In some embodiments, the valve 102 is a hemostasis valve that is configured to maintain hemostasis during a clot treatment procedure by preventing fluid flow in a proximal direction through the valve 102 as various components such as dilators, delivery sheaths, pull members, guidewires, interventional devices, other aspiration catheters, and so on are inserted through the valve 102 to be delivered through the catheter 120 to a treatment site in a blood vessel. The valve 102 can include a branch or side port 104 configured to fluidly couple the lumen 122 of the catheter 120 to the tubing assembly 110. In some embodiments, the valve 102 can be a valve of the type disclosed in U.S. Pat. No. 11,000,682, filed Aug. 30, 2018, and titled “HEMOSTASIS VALVES AND METHODS OF USE,” which is incorporated herein by reference in its entirety.

In the illustrated embodiment, the tubing assembly 110 fluidly couples the catheter 120 to a pressure source 106, such as a syringe. The pressure source 106 can be configured to generate (e.g., form, create, charge, build-up) a vacuum (e.g., negative relative pressure) and store the vacuum for subsequent application to the catheter 120. The tubing assembly 110 can include one or more tubing sections 112 (individually labeled as a first tubing section 112a and a second tubing section 112b), at least one fluid control device 114 (e.g., a valve), and at least one connector 116 (e.g., a Toomey tip connector) for fluidly coupling the tubing assembly 110 to the pressure source 106 and/or other suitable components. In some embodiments, the fluid control device 114 is a stopcock that is fluidly coupled to (i) the side port 104 of the valve 102 via the first tubing section 112a and (ii) the connector 116 via the second tubing section 112b. The fluid control device 114 is externally operable by a user to regulate the flow of fluid therethrough and, specifically, from the lumen 122 of the catheter 120 to the pressure source 106. In some embodiments, the connector 116 is a quick-release connector (e.g., a quick disconnect fitting) that enables rapid coupling/decoupling of the catheter 120 and the fluid control device 114 to/from the pressure source 106.

The system 100 further includes a mechanical thrombectomy assembly 130, shown positioned at least partially within the lumen 122 with a portion thereof extending beyond the distal terminus 124 of the catheter 120. The mechanical thrombectomy assembly 130 can include a proximal or first actuation component 132, a distal or second actuation component 134, one or more embolic protection devices 136, and one or more clot treatment devices 138. In some embodiments, the second actuation component 134 is operably coupled to the embolic protection device 136 via a first catheter or shaft 140. The second actuation component 134 can be an access port or valve, such as hemostasis valve that can include similar or identical features to the valve 102 described in detail above and that can selectively provide access to a lumen of the first shaft 140. The second actuation component 134 can be fluidly coupled to a tubing section 111, additional tubing sections, a fluid control device, a pressure source, etc., to, for example, facilitate aspiration through the first shaft 140. Moving the second actuation component 134, such as moving the second actuation component 134 in a proximal or distal direction, causes a corresponding movement of the embolic protection device 136.

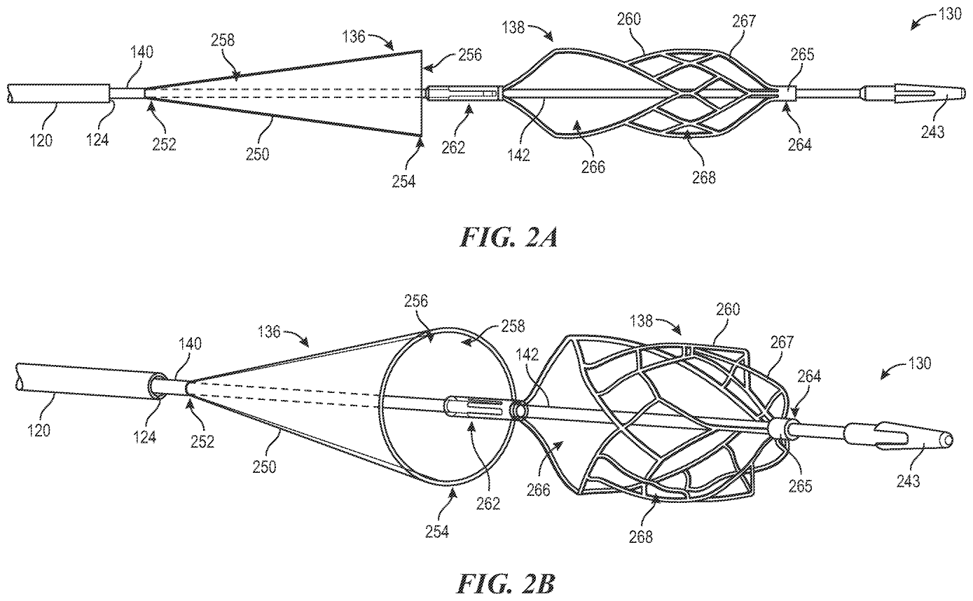

In some embodiments, the first actuation component 132 (e.g., a proximal hub or handle) is operably coupled to the clot treatment device 138 via a second catheter or shaft 142. Accordingly, moving the first actuation component 132, such as moving the first actuation component 132 in a proximal or distal direction, can cause a corresponding movement of the clot treatment device 138. The second shaft 142 can be positioned within and/or extend through the first shaft 140 (e.g., a lumen of the first shaft 140), such that all, or at least a portion, of the first shaft 140 is positioned radially outwardly from the second shaft 142. In some embodiments, at least part of an outer surface of the second shaft 142 can be in sliding contact with an interior or lumen of the first shaft 140. The first actuation component 132 and the second actuation component 134 can be moved in tandem and/or relative to one another, e.g., to insert the embolic protection device 136 and/or the clot treatment device 138 through the catheter 120 and/or distally beyond the distal terminus 124 of the catheter 120. In the illustrated embodiment, the clot treatment device 138 and the embolic protection device 136 are each configured to be deployed from the catheter 120. For example, the catheter 120 can be retracted proximally over the first shaft 140 to deploy the embolic protection device 136, and/or the catheter 120 and/or the first shaft 140 can be retracted proximally over the second shaft 142 to deploy the clot treatment device 138. Such deployment is described in further detail below with reference to FIGS. 2A, 2B, and 6-7E.

During a clot removal procedure, at least a portion of the system 100 including the catheter 120, the embolic protection device 136, and the clot treatment device 138 can be inserted through the vasculature of a patient. In some embodiments, the system 100 is inserted through an introducer sheath that traverses the skin and tissue of the patient to provide an access site. The clot treatment device 138 and/or the embolic protection device 136 can be constrained (e.g., radially-constrained) within the catheter 120 during insertion of the system 100. For example, an interior surface of the catheter 120 can directly contact one or both of the clot treatment device 138 and the embolic protection device 136 to, for example, hold the clot treatment device 138 and/or the embolic protection device 136 in a radially-constrained configuration. The clot treatment device 138 can then be deployed from the catheter 120 (and/or the deployment shaft 141) at least partially distal to clot material in a blood vessel of the vasculature, such as via proximal retraction of the catheter 120. The embolic protection device 136 can then be deployed from the catheter 120 proximal to the clot material, such as via further proximal retraction of the catheter 120. The clot treatment device 138 can engage the clot material to disrupt, capture, and/or core the clot material while the embolic protection device 136 can inhibit or even prevent the clot material from embolizing and/or capture any of the clot material that brakes of (e.g., embolizes) during engagement of the clot treatment device 138 with the clot material and/or redirect all or a portion of the broken-off clot material into the first shaft 140. In some embodiments, the embolic protection device 136 can inhibit or even prevent blood flow past, through, and/or around the embolic protection device 136 within a blood vessel in which the embolic protection device 136 is deployed. Finally, the embolic protection device 136 and the clot treatment device 138 can be withdrawn into the catheter 120, and the clot treatment system 100 can be withdrawn from the patient.

In some embodiments, the catheter 120 can be aspirated during, before, and/or after use of the clot treatment device 138. For example, when the catheter 120 is positioned at a target treatment location proximate to the clot material, a user/operator can first close the fluid control device 114 before generating a vacuum in the pressure source 106 by, for example, withdrawing the plunger of a syringe coupled to the connector 116. In this manner, a vacuum is charged within the pressure source 106 (e.g., a negative pressure is maintained) before the pressure source 106 is fluidly connected to the lumen 122 of the catheter 120. To aspirate the lumen 122 of the catheter 120, the user can open the fluid control device 114 to fluidly connect the pressure source 106 to the catheter 120 and thereby apply or release the vacuum stored in the pressure source 106 to the lumen 122 of the catheter 120. Opening of the fluid control device 114 instantaneously or nearly instantaneously applies the stored vacuum pressure to the tubing assembly 110 and the catheter 120, thereby generating a suction pulse throughout the catheter 120 that can aspirate the clot material into the catheter 120. In some embodiments, the vacuum from the pressure source 106 is applied with the fluid control device 114 in an open position (e.g., to provide continuous vacuum). That is, the user can generate the vacuum in the pressure source 106 while the fluid control device 114 is open (e.g., while the pressure source 106 is fluidly connected to the lumen 122 of the catheter 120) to thereby aspirate the clot material while also simultaneously generating the vacuum, e.g., without or substantially without storing the vacuum in the pressure source 106. In other embodiments, the catheter 120 can be continuously and/or intermittently aspirated via a vacuum pump (e.g., an electric vacuum pump) or other source of aspiration.

Although in FIG. 1A the pressure source 106 is fluidly coupled to the lumen 122 of the catheter 120, in some embodiments the pressure source 106 (or a separate pressure source) is fluidly coupled to a lumen of the first shaft 140 via the tubing section 111 and/or to a lumen of the second shaft 142. Accordingly, the vacuum generated within the pressure source 106 can be applied to the lumen 122 of the catheter 120 and/or the respective lumens of the first shaft 140, the second shaft 142, and/or the deployment shaft 141 to aspirate the clot material.

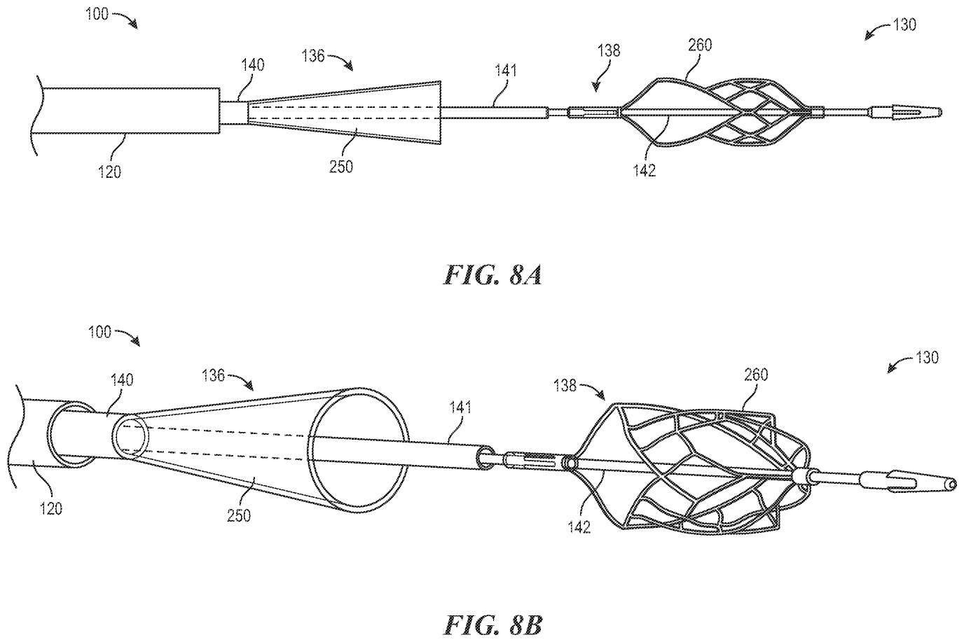

FIG. 1B is a side view of the system 100 configured in accordance with additional embodiments of the present technology. In the illustrated embodiment, the mechanical thrombectomy assembly 130 further includes (i) an additional delivery and/or deployment shaft 141 (which can also be referred to as a “delivery shaft,” an “intermediate shaft,” a “third shaft,” a “catheter,” and/or the like) configured to extend through the first shaft 140 and over the clot treatment device 138 to constrain the clot treatment device 138 and (ii) a third actuation component 139 coupled to the deployment shaft 141 for moving the deployment shaft 141 relative to the clot treatment device 138 to, for example, deploy the clot treatment device 138 from within the deployment shaft 141. That is, the clot treatment device 138 can be positioned within, or at least partially within, the deployment shaft 141 such that the deployment shaft 141 maintains a collapsed or undeployed configuration/state of the clot treatment device 138, and the third actuation component 139 can be withdrawn (e.g., proximally) over and/or relative to the second shaft 142 to deploy the clot treatment device 138. For example, the first actuation component 132 can be pinned in position and the third actuation component 139 can be withdrawn relative to the first actuation component 132. The third actuation component is shown fully withdrawn toward and/or into contact with the first actuation component 132 in FIG. 1B such that the clot treatment device 138 is deployed. Such deployment is described in further detail below with reference to FIGS. 8A-13G. In the illustrated embodiment, the third actuation component 139 is a valve assembly including a connector 135 for attaching to one or more tubing sections, a fluid control device (which can be similar or identical to the fluid control device 114 of FIG. 1A), a pressure source (which can be similar or identical to the pressure source 106 of FIG. 1A), etc., to, for example, facilitate aspiration through the deployment shaft 141.

FIG. 1C is a side view of the system 100 configured in accordance with additional embodiments of the present technology. In the illustrated embodiment, the third actuation component 139 is handle that includes a trigger 144 positioned at least partially within a housing 131. The housing 131 can include a connector 133, e.g., at least generally similar or identical in structure and/or function to the connector 135. The trigger 144 is operably coupled to the deployment shaft 141 via a carriage or slider 145, such that moving the trigger 144 (e.g., proximally or distally) relative to the housing 131 can cause a corresponding movement of the deployment shaft 141. The second shaft 142 extends through the deployment shaft 141 and, in the illustrated embodiment, the first actuation component 132 is coupled (e.g., fixed, pinned) to a proximal portion of the housing 131 such that movement of the trigger 144 moves the deployment shaft 141 relative to the second shaft 142 and the clot treatment device 138. The trigger 144 can include one or more first engagement features or teeth 146, the intermediate actuation component 139 can include one or more second engagement features or recesses 148, and individual ones of the recesses 148 can be configured to receive a corresponding one of the teeth 146. When the teeth 146 are received by and/or contact one or more of the recesses 148, mechanical interference between the teeth 146 and the recesses 148 can inhibit or even prevent movement of the trigger 144 relative to the housing 131. The trigger 144 can be pivotally coupled to the carriage 145 and pivoted (e.g., in the direction shown by arrow P in the enlarged view) to space apart the teeth 146 and the recesses 148, e.g., to allow the trigger 144 to be moved (e.g., slid proximally or distally) relative to the housing 131. In some embodiments, the trigger 144 can be biased (e.g., via a spring) to pivot opposite the direction indicated by the arrow P such that the teeth 146 engage the recesses 148 to inhibit or even prevent movement of the trigger absent an external force (e.g., a force applied by a finger of an operator/user). In some aspects of the present technology, the configuration of the third actuation component 139 can allow for more precise deployment of the clot treatment device 138 from the deployment shaft 141.

FIGS. 2A and 2B are a side view and a perspective view, respectively, of a distal portion of the clot treatment system 100 of FIG. 1A in accordance with embodiments of the present technology. In the illustrated embodiment, the embolic protection device 136 includes a conical member or funnel 250 having a first or proximal end portion 252 and a second or distal end portion 254 opposite the proximal end portion 252. In these and/or other embodiments, the embolic protection device 136 can include a balloon, a disc, a braided structure, and/or another suitable embolic protection device 136. In the illustrated embodiment, the funnel 250 includes a conically-shaped polymer layer. The funnel 250 can additionally or alternatively include a coil, one or more wires a mesh, a braided structure, an at least partially coated and/or dipped structure, and/or another suitable structure. The coils, wires, mesh, etc., used to form the funnel 250 can be radiopaque and/or can be coated with a radiopaque material, e.g., to improve imaging/visualization of the funnel 250 within the patient. In these and/or other embodiments, the funnel 250 can be configured to inhibit or even prevent fluid (e.g., blood) flow (e.g., through one or more surfaces of the funnel 250) when deployed. That is, the funnel 250 can be impermeable to blood flow. For example, in some embodiments the funnel 250 includes a wire braid that has been dip-coated to form an at least partially fluid-impermeable membrane around all, or at least a portion, of the wire braid. The distal end portion 254 of the funnel 250 can define an opening 256 to an interior 258 of the funnel 250. A diameter (e.g., an outer diameter and/or an inner diameter) of the funnel 250 can increase from a first diameter at the proximal end portion 252 to a second diameter greater than the first diameter at the distal end portion 254. At least part of the funnel 250 (e.g., the proximal end portion 252) can be coupled to the first shaft 140, such as a distal terminus of the first shaft 140. Accordingly, all or at least a portion of the fluid that enters the interior 258 of the funnel 250 (e.g., via passive flow, during aspiration, etc.) can be directed into a lumen of the first shaft 140.

The funnel 250 can be self-expanding and configured to transition from a collapsed or undeployed configuration to an expanded or deployed configuration (shown in FIGS. 2A and 2B). For example, the funnel 250 can be in the collapsed configuration when positioned within a larger delivery catheter, such as the catheter 120, and the funnel 250 can expand to the expanded configuration when the catheter 120 (or other delivery catheter) is retracted and/or the first shaft 140 is advanced so that the funnel 250 is no longer constrained by the catheter 120, e.g., positioned distal of the distal terminus 124 of the catheter 120.

The clot treatment device 138 illustrated in FIGS. 2A and 2B has a different configuration than the clot treatment device 138 shown in FIGS. 1A-1C. In the illustrated embodiment, the clot treatment device 138 includes an expandable clot removal element 260 coupled to the second shaft 142 that extends through the first shaft 140. For example, the clot removal element 260 can be a single (e.g., unitary) laser cut metal (e.g., nitinol) element formed from a plurality of interconnected struts 267 configured to self-expand. The clot removal element 260 has a proximal end portion 262 and a distal end portion 264. The proximal end portion 262 can be fixed to the second shaft 142 and the distal end portion 264 can include (e.g., be joined at) a hub or tube 265 that is slidable over the second shaft 142. In other embodiments, the proximal end portion 262 can be slidable over the second shaft 142 and the distal end portion 264 can be fixed to the second shaft, both the proximal end portion 262 and the distal end portion 264 can be fixed to the second shaft 142, or both the proximal end portion 262 and the distal end portion 264 can be slidably positioned over the second shaft 142. A proximal portion of the clot removal element 260 can include/define one or more proximal mouths or cells 266 (e.g., relatively larger open cells formed/defined by the struts 267) and a distal portion of the clot removal element 260 can be relatively more closed (e.g., including smaller open cells 268 formed/defined by the struts 267). In some aspects of the present technology, the slidable distal end portion 264 allows the clot removal element 260 to longitudinally lengthen/shorten and to correspondingly radially collapse/expand.

The clot removal element 260 can be self-expanding and configured to transition from a collapsed or undeployed configuration to an expanded or deployed configuration (shown in FIGS. 2A and 2B). For example, the clot removal element 260 can be in the collapsed configuration when positioned within a larger delivery catheter, such as the delivery catheter 120, and the clot removal element 260 can expand to the expanded configuration when the delivery catheter 120 (or other delivery catheter) is retracted and/or the second shaft 142 is advanced so that the clot removal element 260 is no longer constrained by the first shaft 140 and/or the funnel 250, e.g., when positioned distal of a distal terminus of the first shaft 140 and/or distal of the opening 256 of the funnel 250.

In some embodiments, the second shaft 142 includes an atraumatic tip 243 defining a distalmost terminus of the mechanical thrombectomy assembly 130. The distal end portion 264 of the clot removal clement 260 can contact at least part of the atraumatic tip 243 when the clot removal element 260 is in the collapsed configuration.

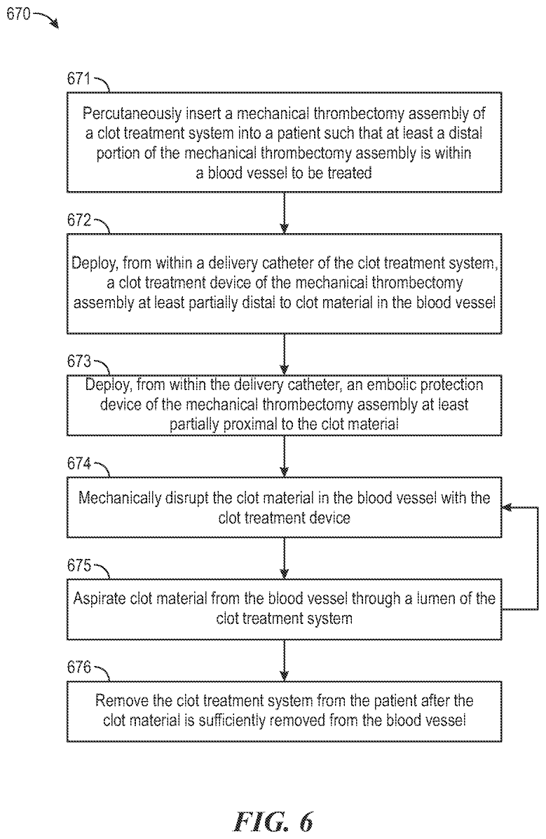

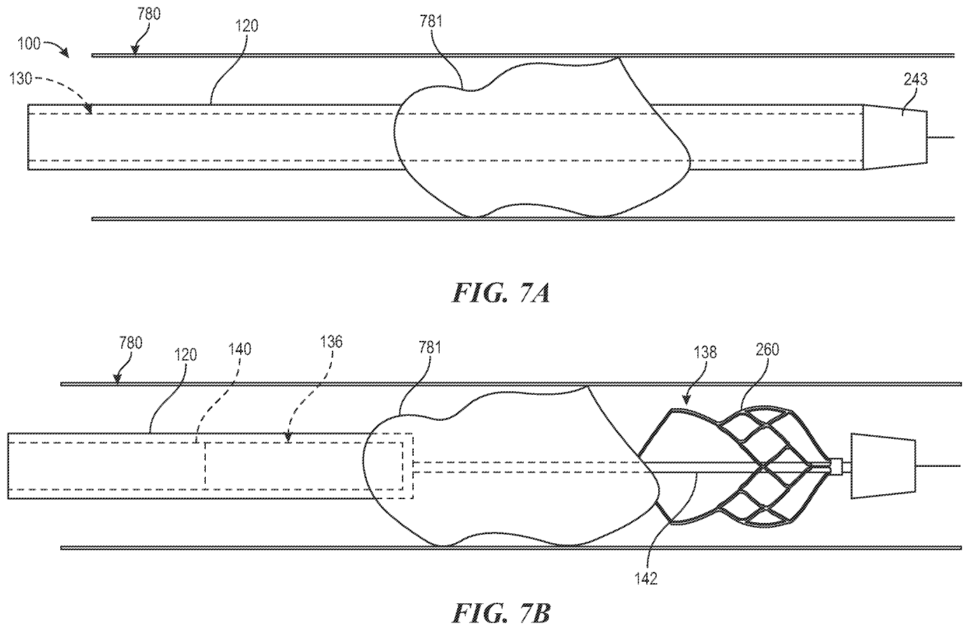

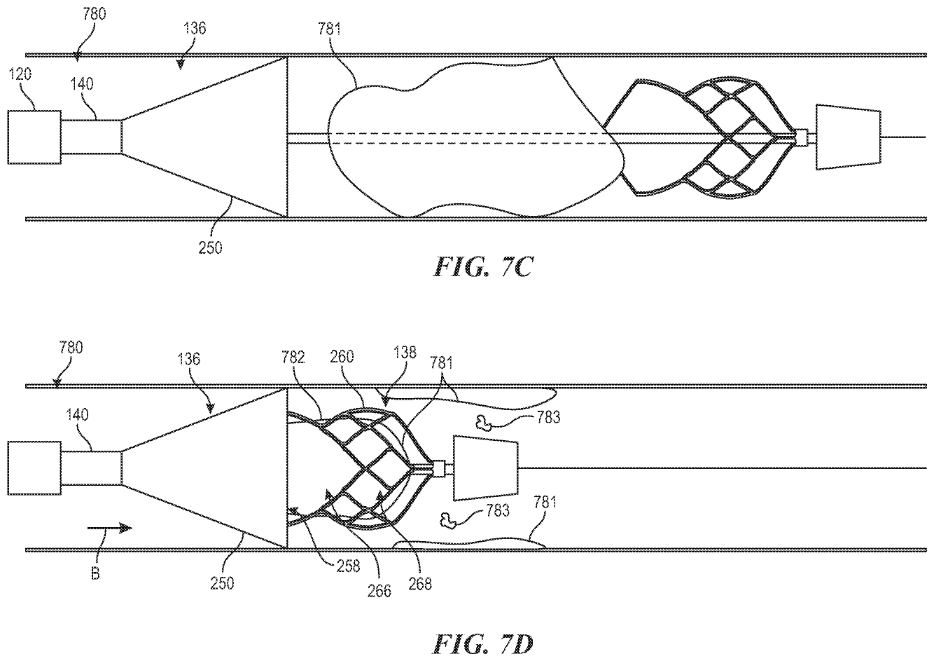

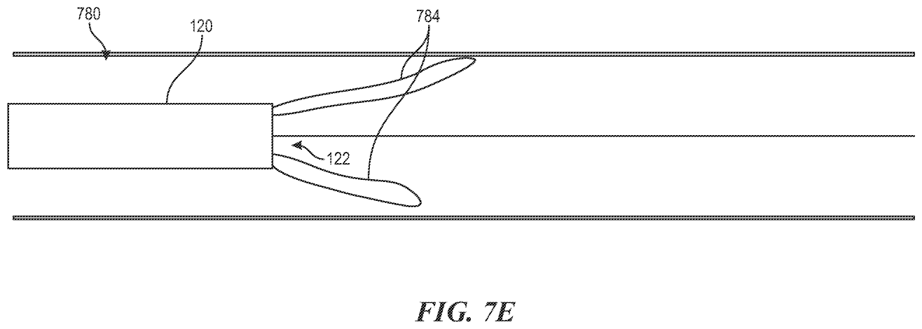

As described in greater detail below with reference to FIGS. 6-7E, the same delivery catheter (e.g., the catheter 120) can be used to deploy both the clot treatment device 138 and the embolic protection device 136. For example, the catheter 120 can be retracted proximally to unsheathe and deploy the clot treatment device 138 and the embolic protection device 136. Accordingly, the assembly 130 is expected to have a reduced profile or outer diameter compared to other mechanical thrombectomy assemblies that use another catheter or shaft positioned between the first shaft 140 and the second shaft 142 to deploy the clot treatment device 138. In such embodiments, the system 100 can be an integrated system for both mechanical thrombectomy and embolic protection.

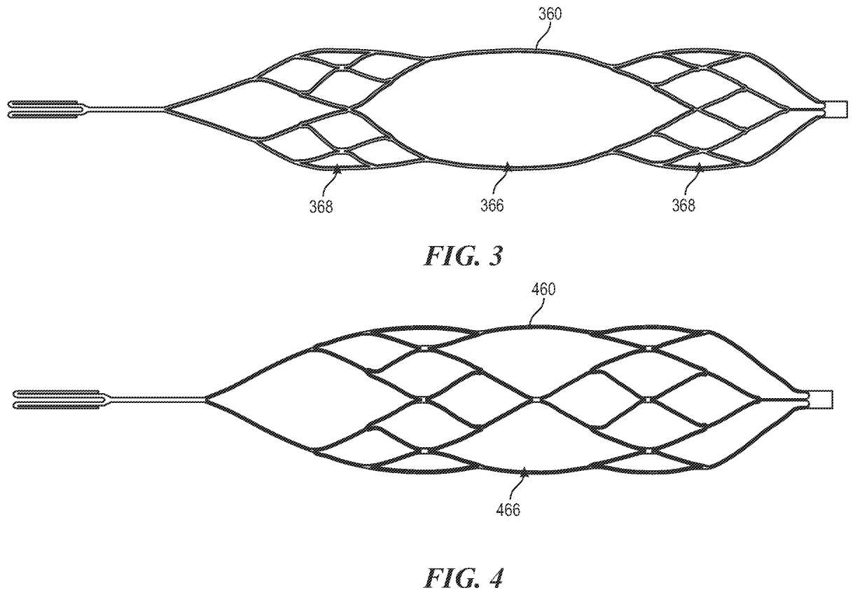

FIGS. 3 and 4 are side views of respective clot removal elements 360, 460 configured in accordance with additional embodiments of the present technology. Referring to FIG. 3, the clot removal element 360 is at least generally similar in structure and/or function to the clot removal element 260 of FIGS. 2A and 2B, but includes smaller open cells 368 positioned proximally and distally of one or more larger open cells 366. Referring to FIG. 4, the clot removal element 460 is at least generally similar in structure and/or function to the clot removal element 360 of FIG. 3, but includes large open cells 466 of smaller dimension compared to the larger open cells 366 of FIG. 3. In some embodiments, the clot removal element 260, the clot removal clement 360, and/or the clot removal element 360 can include some features generally similar or identical, and can function in a generally similar or identical manner, to any of the clot removal elements described in detail U.S. patent application Ser. No. 17/072,909, titled “SYSTEMS, DEVICES, AND METHODS FOR TREATING VASCULAR OCCLUSIONS,” filed Oct. 16, 2020, and U.S. patent application Ser. No. 17/125,397, titled “DEVICES AND METHODS FOR TREATING VASCULAR OCCLUSION,” filed Dec. 17, 2020, the each of which is hereby incorporated by reference in its entirety.

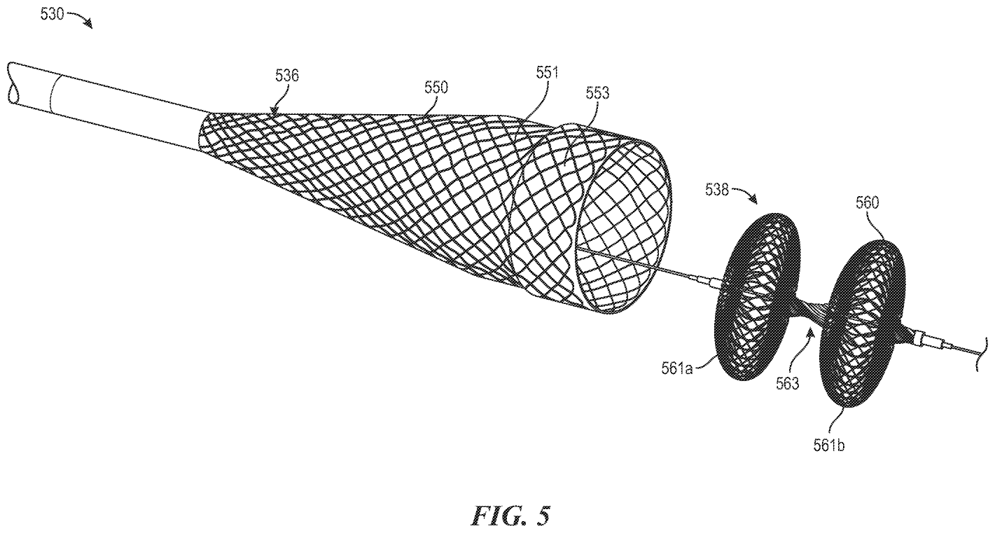

FIG. 5 is a perspective view of a mechanical thrombectomy assembly 530 (“assembly 530”) configured in accordance with additional embodiments of the present technology. At least some aspects of the assembly 530 can be at least generally similar or identical in structure and/or function to the mechanical thrombectomy assembly 130 of FIGS. 1A, 2A, and 2B. For example, the mechanical thrombectomy assembly 530 includes an embolic protection device 536 including a funnel 550 and a clot treatment device 538 including a clot removal element 560. The funnel 550 includes a braided structure 551 and a material layer 553 coupled to the braided structure 551 (e.g., within the braided structure 551). The braided structure 551 can be formed from nitinol and/or other suitable materials. In some embodiments, the material layer 553 can be impermeable, or at least generally impermeable, to clot material, blood, fluid, emboli, and/or other material. The braided structure 551 can be deformable (e.g., configured to deform elastically). In at least some embodiments, the funnel 550 can be deployed from and/or retracted into another funnel and/or conically-shaped structure, e.g., configured to constrain the braided structure 551 and/or the material layer 553 in a collapsed or delivery configuration. The clot removal element 560 includes a plurality of expandable discs or disc portions 561 (individually identified as a first or proximal disc portion 561a and a second or distal disc portion 561b) and a narrowed portion 563 positioned between individual ones of the discs 561. In some embodiments, the clot removal element 560 can be at least generally similar or identical in structure and/or function to one or more of the clot treatment devices described in U.S. Pat. No. 10,709,471, titled “METHODS AND APPARATUS FOR TREATING EMBOLISM,” and filed Apr. 10, 2018, which is incorporated herein by reference in its entirety.

FIG. 6 is a flow diagram of a process or method 670 for removing clot material from the vasculature of a patient using a clot treatment system in accordance with embodiments of the present technology. Although some features of the method 670 are described in the context of the clot treatment system 100 shown in FIGS. 1A, 2A, and 2B for illustration, one skilled in the art will readily understand that the method 670 can be carried out using other suitable systems and/or devices described herein, including the clot removal element 360 of FIG. 3, the clot removal element 460 of FIG. 4, and/or the mechanical thrombectomy assembly 530 of FIG. 5. FIGS. 7A-7E are partially schematic side views of a distal portion of the clot treatment system 100 of FIGS. 1A, 2A, and 2B during different stages of the method 670, in accordance with embodiments of the present technology. Individual ones of FIGS. 7A-7E are described below with reference to a one or more blocks 671-676 of the method 670.

At block 671, the method 670 can include percutaneously inserting a mechanical thrombectomy assembly of a clot treatment system into a patient such that at least a distal portion of the mechanical thrombectomy assembly is positioned within a blood vessel to be treated. For example, FIG. 7A shows the catheter 120 interested through the skin of a patient and into a blood vessel 780 of the patient including clot material 781 to be treated. The blood vessel 780 can be an artery within the peripheral vasculature. The mechanical thrombectomy assembly 130 is constrained/collapsed within the catheter 120. As such, the clot treatment system 100 can be an integrated thrombectomy and embolic protection system in which the clot treatment device 138 and the embolic protection device 136 can be delivered together within the catheter 120 to the clot material 781.

In some embodiments, at least part of the assembly 130 can be positioned within or extend distally beyond the clot material 781 such that, for example, the atraumatic tip 243 is inserted entirely through the clot material 781. To position the assembly 130 at least proximate to and/or at least partially through the clot material 781, the first and second actuation components 132, 134 (FIG. 1A) can be advanced together along with the catheter 120 into and/or through the blood vessel 780. In some instances, the clot material 781 can substantially occlude the blood vessel 780, such as shown in FIG. 7A, or can partially occlude the blood vessel 780.

At block 672, the method 670 can include deploying, from within a delivery catheter of the clot treatment system, a clot treatment device of the mechanical thrombectomy assembly at least partially distal to clot material in the blood vessel. For example, FIG. 7B shows the clot treatment device 138 including the clot removal element 260 deployed/expanded within the blood vessel 780, with at least a portion of the clot removal element 260 positioned distal to the clot material 781. The clot treatment device 138 can be deployed/expanded by retracting (e.g., proximally) the catheter 120. Additionally, or alternatively, the clot treatment device 138 can be deployed/expanded by depressing the trigger 144 of the first actuation component 132 (FIG. 1A) and advancing the trigger 144 and the carriage 145 distally, e.g., to move the second shaft 142 (e.g., distally) relative to the catheter 120 to position the clot treatment device 138 at least partially outside the catheter 120. The embolic protection device 136 can remain undeployed/packaged within the catheter 120 while the clot treatment device 138 is deployed in the blood vessel 780.

At block 673, the method 670 can include deploying, from within the delivery catheter, an embolic protection device of the mechanical thrombectomy assembly at least partially proximal to the clot material in the blood vessel. For example, FIG. 7C shows the embolic protection device 136 including the funnel 250 deployed/expanded within the blood vessel 780, with at least a portion of the funnel 250 positioned proximally of the clot material 781. The embolic protection device 136 can be deployed/expanded by retracting (e.g., proximally) the catheter 120. Additionally, or alternatively, the embolic protection device 136 can be deployed/expanded by advancing the second actuation component 134 distally, e.g., to move the first shaft 140 (e.g., distally) relative to the catheter 120 to position the embolic protection device 136 at least partially outside the catheter 120. When expanded/deployed, the embolic protection device 136 can span all or a portion of the width/diameter of the blood vessel 780. Notably, referring to blocks 672 and 673 together, the outer delivery catheter 120 can be retracted proximally (e.g., in stages or in a continuous motion) to deploy both the clot treatment device 138 and the embolic protection device 136. When expanded, the embolic protection device 136 can substantially or entirely block blood flow through the blood vessel 780.

At block 674, the method 670 can include mechanically disrupting the clot material in the blood vessel with the clot treatment device. For example, FIG. 7D shows the clot treatment device 138 (e.g., the clot removal element 260) retracted (e.g., proximally) to capture at least a portion 782 of the clot material 781. The proximal mouths 266 of the clot removal element 260 can receive at least the portion 782 of the clot material 781 and the relatively more closed distal portion (e.g., smaller open cells 268) of the clot removal element 260 can retain the received portion 782 within, or at least partially within, the clot removal element 260. The clot treatment device 138 can be retracted through/against the clot material 781 toward the embolic protection device 136 and/or at least partially into the interior 258 of the funnel 250. For example, the trigger 144 can be depressed and the trigger 144 and the carriage 145 can be withdrawn proximally relative to the housing of the first actuation component 132 to pull the clot removal element 260 through the clot material 781, toward the embolic protection device 136, and/or at least partially into the interior 258 of the funnel 250. Additionally, or alternatively, the trigger 144 can be locked to the housing of the first actuation component 132 (e.g., by mechanical interference between the tooth 146 and the recesses 148 when the tooth 146 is positioned in contact with one of the recesses 148), and the first actuation component 132 can be withdrawn proximally relative to the housing of the first actuation component 132 to pull the clot removal element 260 through the clot material 781, toward the embolic protection device 136, and/or at least partially into the interior 258 of the funnel 250.

The embolic protection device 136 (e.g., the funnel 250) is positioned proximal to the clot material 781 and configured to direct the clot removal element 260 and the received portion 782 contained within the clot removal element 260 into the first shaft 140. Blood in the blood vessel 780 can flow in the direction indicated by arrow B. However, when deployed proximal to the clot material 781, the funnel 250 can inhibit or even prevent blood from flowing distally through and/or past the funnel 250 and past the clot material 781. In some aspects of the present technology, such arrest of blood flow provided by the funnel 250 is expected to inhibit or even prevent the clot material fragments 783 from embolizing and flowing downstream in the direction of arrow B further into the vasculature of the patient. In some embodiments, after the clot material 781 is sufficiently disrupted, block 674 can include removing the mechanical thrombectomy assembly 130 from the patient, e.g., by retracting (e.g., proximally) one or both of the first shaft 140 and the second shaft 142 into and/or through the catheter 120.

In some embodiments, at block 675, the method 670 can include aspirating clot material from the blood vessel through a lumen of the clot treatment system 100. The aspirated clot material can include all or a portion of the clot material remaining in the blood vessel after the mechanical disruption in block 674. For example, FIG. 7E shows remaining clot material 784 being aspirated from the blood vessel 780 through the lumen 122 of the catheter 120. In other embodiments, the lumen of the first shaft 140 and/or the lumen of the second shaft 142 can be used to aspirate material from the blood vessel 780. In some embodiments, blocks 674 and 675 can be repeated until all, or at least a sufficient quantity, of the material in the vasculature has been removed, via one or both of mechanical disruption (e.g., block 674) and aspiration (e.g., block 675). The sufficient quantity can be at least 50%, 60%, 70%, 80%, 90%, 95%, 99%, etc., of the clot material at a given location in the patient. Although block 675 is shown as occurring after block 674 in FIG. 6, in other embodiments block 675 can occur before block 674 and/or at a same time as block 674. After the clot material has been sufficiently removed, the method 670 can include removing the clot treatment system 100 from the patient (block 676).

FIGS. 8A and 8B are a side view and a perspective view, respectively, of a distal portion of the clot treatment system 100 of FIGS. 1B and/or 1C configured in accordance with additional embodiments of the present technology. In the illustrated embodiment, the mechanical thrombectomy assembly 130 further includes the (e.g., intermediate) deployment shaft 141. The deployment shaft 141 can be positioned at least partially between (e.g., radially between) the first shaft 140 and the second shaft 142. In the illustrated embodiment, for example, the deployment shaft 141 is positioned within the first shaft 140 and configured to slidably receive the second shaft 142. The deployment shaft 141 can be positioned over the clot removal element 260 to collapse and constrain the clot removal element 260 in the collapsed configuration. Accordingly, during a clot removal procedure, the deployment shaft 141 can be retracted proximally relative to the clot removal element 260 (and/or the clot removal element 260 can be advanced distally relative to the deployment shaft 141) to unsheathe and deploy the clot removal element 260. The catheter 120 can then be withdrawn to unsheathe and deploy the funnel 250 (as described previously herein). In such embodiments, the system 100 is systemized for both mechanical thrombectomy and embolic protection.

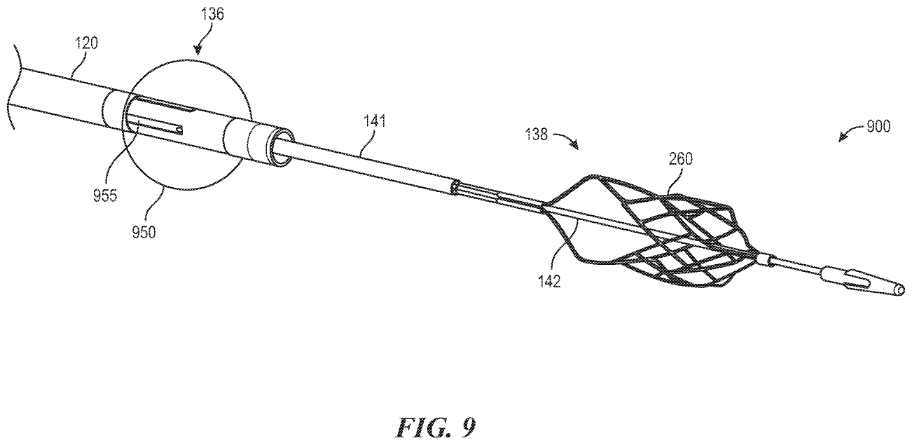

FIG. 9 is a perspective view of a distal portion of a clot treatment system 900 configured in accordance with embodiments of the present technology. At least some aspects of the clot treatment system 900 can be at least generally similar or identical in structure and/or function to the clot treatment system 100 of FIGS. 1B-2B, 8A, and 8B. For example, the clot treatment system 900 includes the clot treatment device 138, the deployment shaft 141, the second shaft 142, and the clot removal element 260 described in detail previously herein. However, in the illustrated embodiment the embolic protection device 136 comprises a balloon 950 carried by the catheter 120 and configured to inflate radially outwardly from the catheter 120. Accordingly, the first shaft 140 and the funnel 250 can be omitted. One or more inflation lumens 955 for the balloon 950 can be positioned at least partially within a sidewall of the catheter 120 for inflating/deflating the balloon 950 concentrically. The deployment shaft 141 can be positioned over the clot removal element 260 to collapse and constrain the clot removal element 260 in the collapsed configuration. Accordingly, during a clot removal procedure, the deployment shaft 141 can be retracted proximally relative to the clot removal element 260 (and/or the clot removal element 260 can be advanced distally relative to the deployment shaft 141) to unsheathe and deploy the clot removal element 260. The balloon 950 can be inflated to provide embolic protection during use of the clot removal element 260. In such embodiments, the system 100 is systemized for both mechanical thrombectomy and embolic protection.

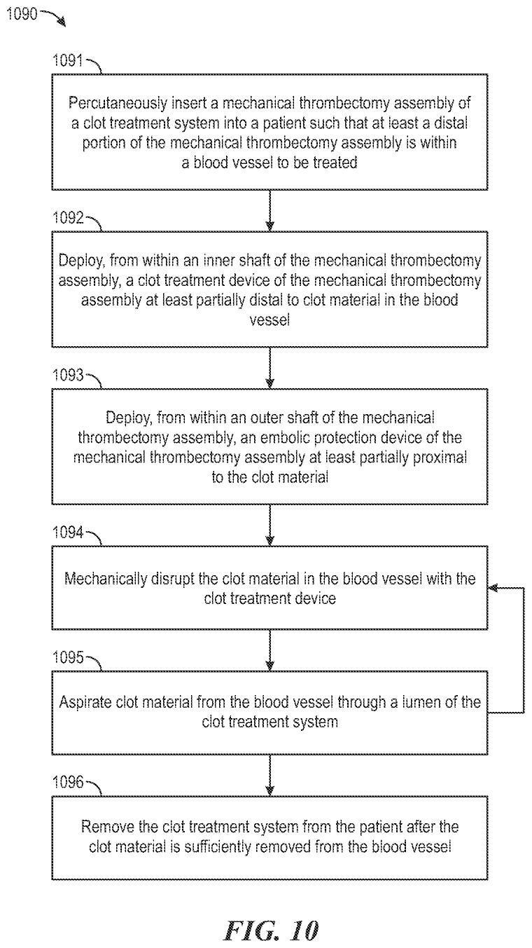

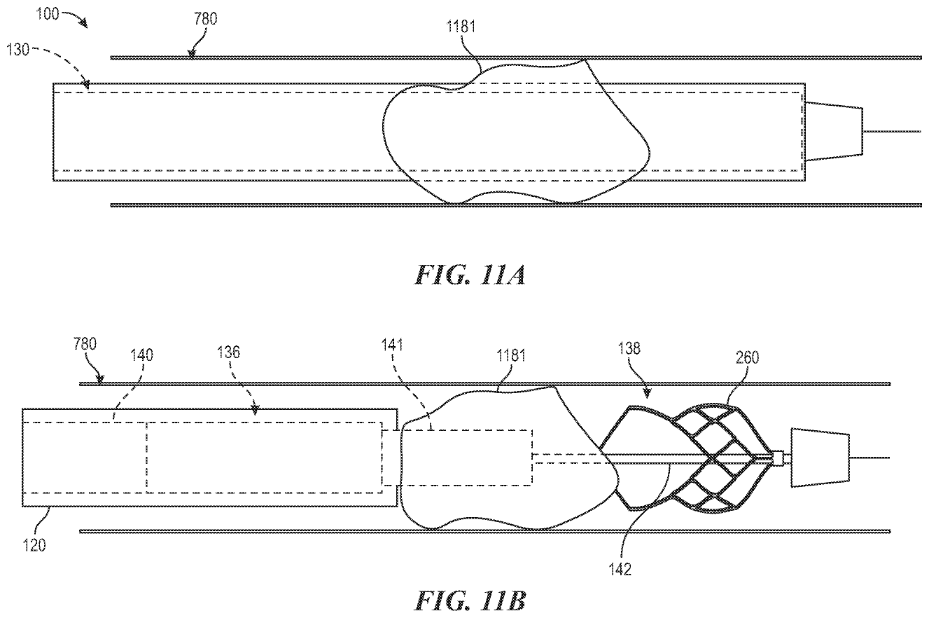

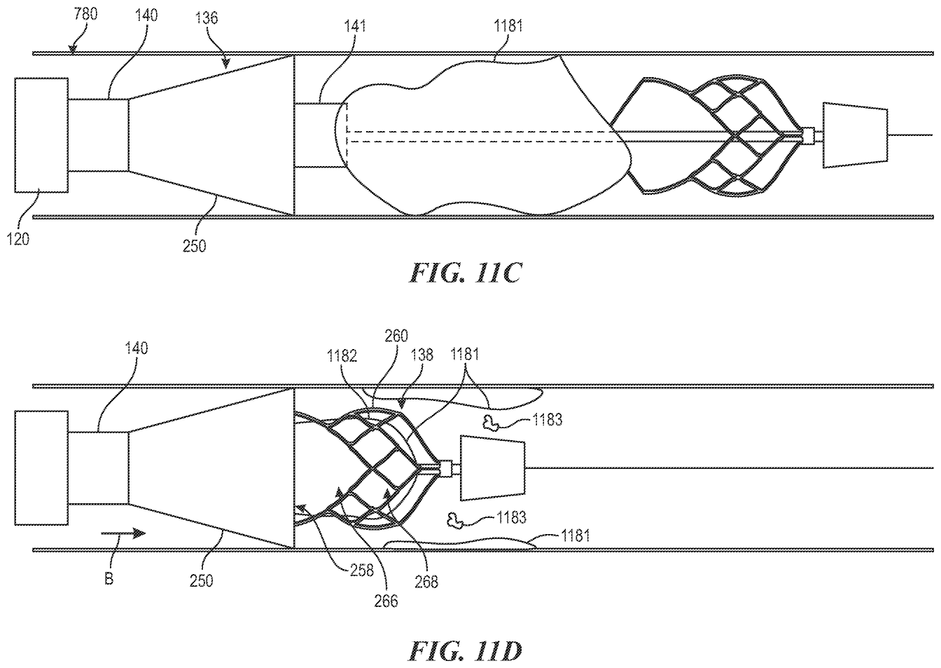

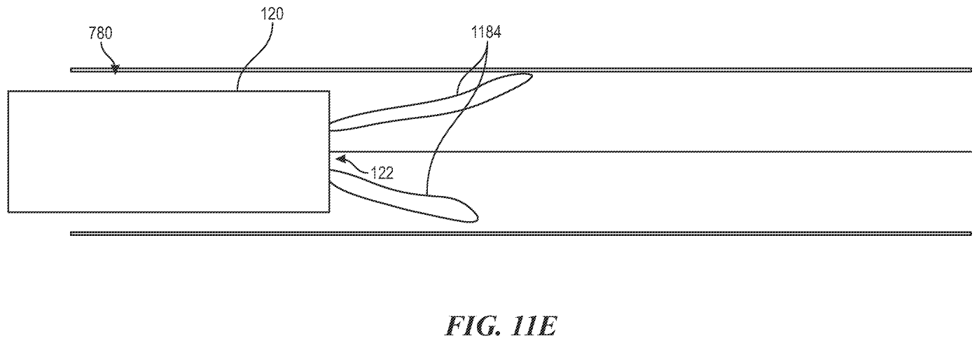

FIG. 10 is a flow diagram of a process or method 1090 for removing clot material from the vasculature of a patient using a clot treatment system in accordance with embodiments of the present technology. Although some features of the method 1090 are described in the context of the embodiments shown in FIGS. 8A and 8B for illustration, one skilled in the art will readily understand that the method 1090 can be carried out using other suitable systems and/or devices described herein, such as the system 900 of FIG. 9. FIGS. 11A-11E are partially schematic side views of a distal portion of the clot treatment system 100 of FIGS. 8A and 8B during different stages of the method 1090, in accordance with embodiments of the present technology. Individual ones of FIGS. 11A-11E are described below with reference to one or more blocks 1091-1096 of the method 1090.

At block 1091, the method 1090 can include percutaneously inserting a mechanical thrombectomy assembly of a clot treatment system into a patient such that at least a distal portion of the mechanical thrombectomy assembly is within a blood vessel to be treated. For example, FIG. 11A shows the mechanical thrombectomy assembly 130 interested through skin of a patient and into the blood vessel 780 of the patient including clot material 1181 to be treated. The mechanical thrombectomy assembly 130 is constrained/collapsed within the catheter 120. As such, the clot treatment system 100 can be a systemized thrombectomy and embolic protection system in which the clot treatment device 138 and the embolic protection device 136 can be delivered together within the catheter 120. In some embodiments, at least part of the assembly 130 can be positioned within or extend distally beyond the clot material 1181. In some instances, the clot material 1181 can substantially occlude the blood vessel 780, such as shown in FIG. 11A, or can partially occlude the blood vessel 780.

At block 1092, the method 1090 can include deploying, from within an inner shaft of the mechanical thrombectomy assembly, a clot treatment device of the mechanical thrombectomy assembly at least partially distal to clot material in the blood vessel. For example, FIG. 11B shows the clot treatment device 138 including the clot removal element 260 deployed/expanded in the blood vessel 780 from within the deployment shaft 141, with at least a portion of the clot removal element 260 positioned distal to the clot material 1181. The clot treatment device 138 can be deployed/expanded by retracting (e.g., proximally) the deployment shaft 141 and/or by advancing the second shaft 142 (e.g., distally) within the deployment shaft 141 after retracting the catheter 120. The embolic protection device 136 can remain undeployed/packaged within the catheter 120 while the clot treatment device 138 is deployed in the vasculature.

At block 1093, the method 1090 can include deploying, from within an outer shaft of the mechanical thrombectomy assembly, an embolic protection device of the mechanical thrombectomy assembly at least partially proximal to the clot material. For example, FIG. 11C shows the embolic protection device 136 including the funnel 250 deployed/expanded in the blood vessel 780 from within the catheter 120, with at least a portion of the funnel 250 positioned proximally of the clot material 1181. The embolic protection device 136 can be deployed/expanded by retracting (e.g., proximally) the catheter 120. In some embodiments, deploying the embolic protection device 136 includes inflating a balloon, such as the balloon 950 of FIG. 9. Accordingly, in some aspects of the present technology the embolic protection device 136 is deployed via a separate mechanism (e.g., via the catheter 120 or via expansion of balloon) than the clot treatment device 138 while still forming part of a systemized clot treatment system that can be advanced and used together. When expanded/deployed, the embolic protection device 136 can span all or a portion of the width/diameter of the blood vessel 780, and can prevent or substantially prevent blood flow through the blood vessel 780 (e.g., arrest blood flow therethrough). Although block 1093 is shown and described as occurring after block 1092, in some embodiments block 1093 can occur prior to block 1092. For example, in some embodiments the method 1090 includes deploying the embolic protection device (e.g., block 1093, FIG. 11C) and then deploying the clot treatment device (e.g., block 1092, FIG. 11B).

At block 1094, the method 1090 can include mechanically disrupting the clot material in the blood vessel with the clot treatment device. For example, FIG. 11D shows the clot treatment device 138 retracted (e.g., proximally) to capture at least a portion 1182 of the clot material 1181. The proximal mouths 266 of the clot removal element 260 can receive at least the portion 1182 of the clot material 1181 and the relatively more closed distal portion (e.g., smaller open cells 268) of the clot removal element 260 can retain the received portion 1182 within, or at least partially within, the clot removal element 260. The clot treatment device 138 can be retracted toward and through the embolic protection device 136 and/or at least partially into the interior 258 of the funnel 250. As described in detail above with reference to FIGS. 6-7E, the embolic protection device 136 (e.g., the funnel 250 and/or the balloon 950) is positioned proximal to the clot material 1181 to inhibit or even prevent blood flow (in the direction shown by the arrow B) through the blood vessel 780. In some aspects of the present technology, blocking blood flow through the blood vessel 780 via the embolic protection device 136 can inhibit clot material fragments 1183 that break off from the clot material 1181 from migrating downstream farther into the vasculature of the patient. Additionally, the embolic protection device 136 can direct the clot removal element 260 and the received portion 1182 contained within the clot removal element 260 into the first shaft 140. In some embodiments, after the clot material 1181 is sufficiently disrupted, block 1094 can include removing the mechanical thrombectomy assembly 130 from the patient, e.g., by retracting (e.g., proximally) one or both of the first shaft 140, the second shaft 142 (FIG. 11B), and/or the deployment shaft 141 (FIG. 11C).

In some embodiments, at block 1095, the method 1090 can include aspirating clot material from the blood vessel through a lumen of the clot treatment system. The aspirated clot material can include any of the clot material 1181 remaining after the mechanical disruption in block 1094. For example, FIG. 11E shows remaining clot material 1184 being aspirated from the blood vessel 780 through the lumen 122 of the catheter 120. In other embodiments, aspiration can be applied through the lumen of the first shaft 140, the lumen of the second shaft 142, and/or the lumen of the deployment shaft 141 to aspirate the clot material 1181. In some embodiments, blocks 1094 and 1095 can be repeated until all, or at least a sufficient quantity, of the clot material in the vasculature has been removed, via one or both of mechanical disruption (e.g., in block 1094) and aspiration (e.g., in block 1095). Although block 1095 is shown as occurring after block 1094 in FIG. 10, in other embodiments block 1095 can occur before block 1094 and/or at a same time as block 1094. After the clot material has been sufficiently removed, the method 1090 can include removing the clot treatment system 100 from the patient (block 1096).

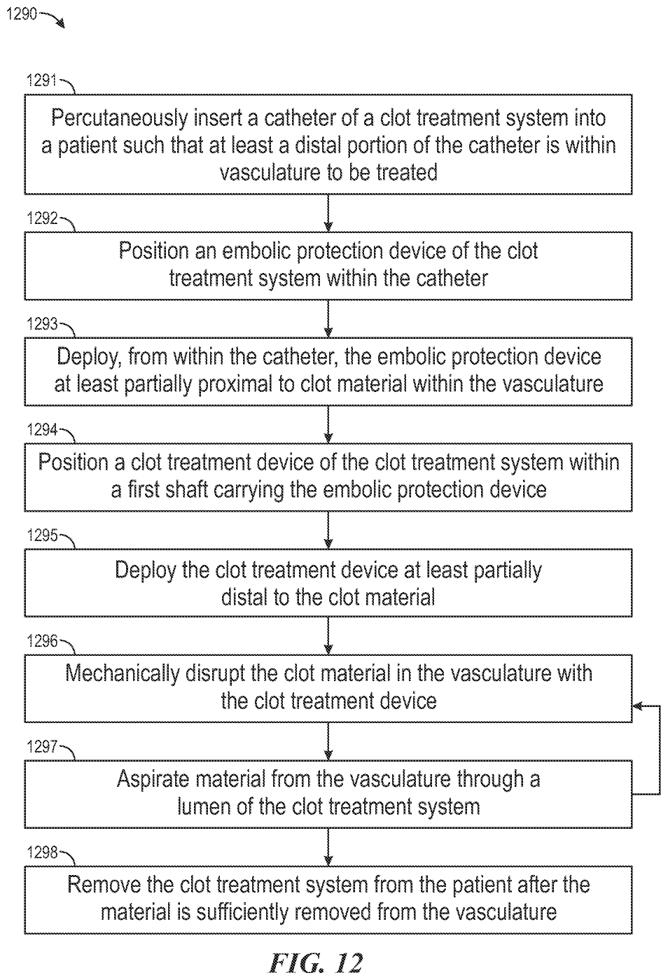

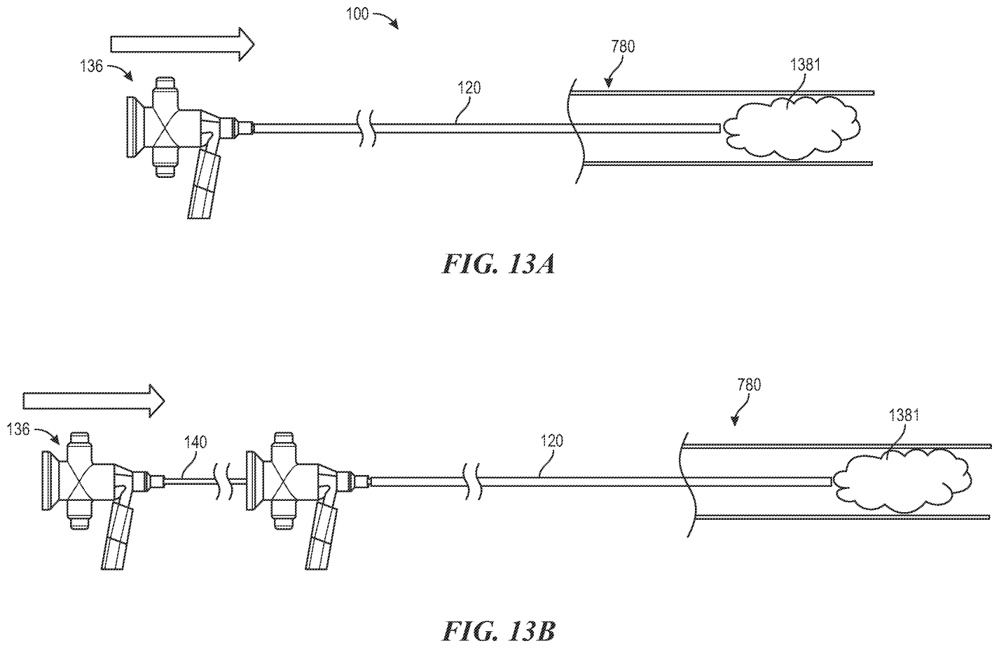

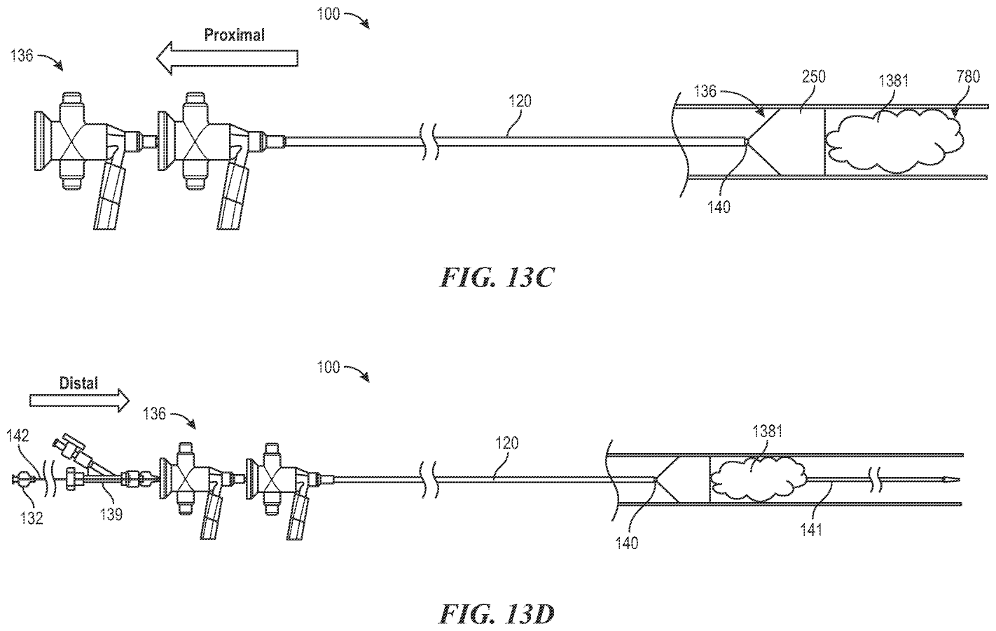

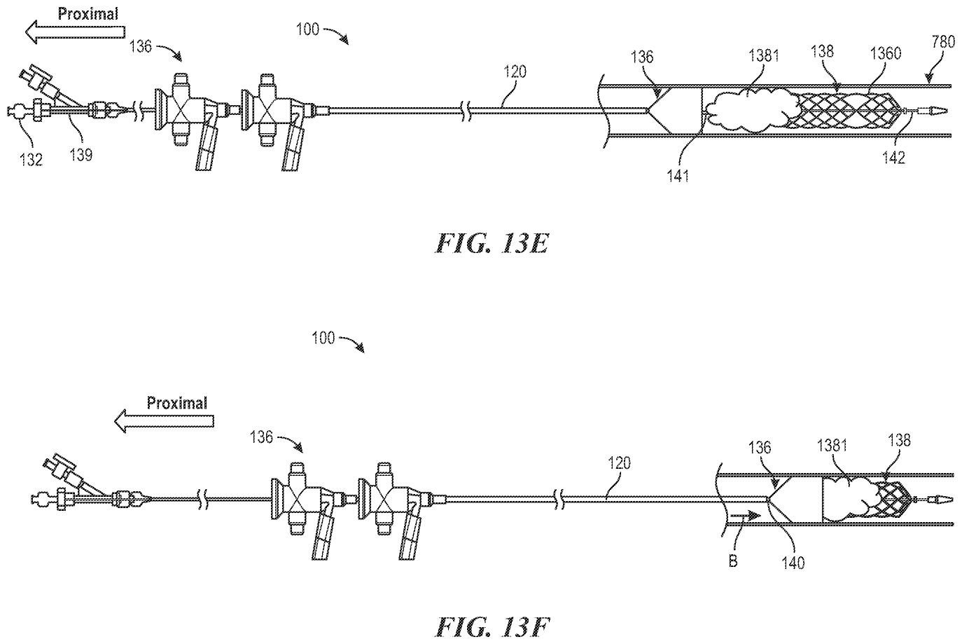

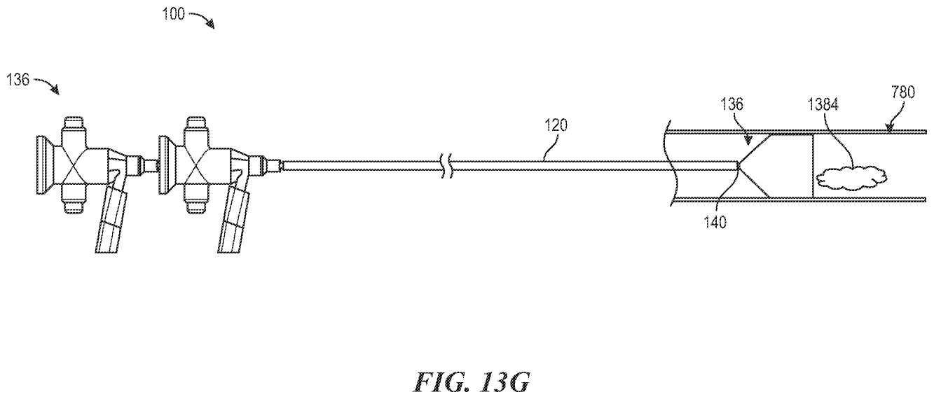

FIG. 12 is a flow diagram of a process or method 1290 for removing clot material from the vasculature of a patient using a clot treatment system in accordance with embodiments of the present technology. At least some aspects of the method 1290 are at least generally similar or identical to one or more of the blocks 1090-1096 of the method 1090. Although some features of the method 1290 are described in the context of the clot treatment system 100 shown in FIGS. 1B, 8A, and 8B for illustration, one skilled in the art will readily understand that the method 1290 can be carried out using other suitable systems and/or devices described herein, such as the system 900 of FIG. 9. FIGS. 13A-13G are side views of the clot treatment system 100 of FIGS. 1B, 8A, and 8B during different stages of the method 1290, in accordance with embodiments of the present technology. Individual ones of FIGS. 13A-13G are described below with reference to one or more blocks 1291-1298 of the method 1290.

At block 1291, the method 1290 can include percutaneously inserting a catheter of a clot treatment system into a patient such that at least a distal portion of the catheter is positioned within a blood vessel to be treated. For example, FIG. 13A shows the catheter 120 interested through the skin of a patient and into the blood vessel 780 including clot material 1381 to be treated. In some instances, the clot material 1381 can substantially occlude the blood vessel 780, such as shown in FIG. 13A, or can partially occlude the blood vessel 780. Block 1291 can be at least generally similar to block 1091 and/or the method/process described with reference to FIG. 11A.

At block 1292, the method 1290 can include positioning an embolic protection device of the clot treatment system within the catheter. For example, FIG. 13B shows the first shaft 140 positioned within the catheter 120. The first shaft 140 can carry the embolic protection device 136 (FIGS. 8A and 8B) so that both can be advanced distally through the catheter toward the clot material 1181.

At block 1293, the method 1290 can include deploying, from within the catheter, the embolic protection device at least partially proximal to the clot material in the blood vessel. For example, FIG. 13C shows the embolic protection device 136 including the funnel 250 deployed/expanded within the blood vessel 780, with at least a portion of the funnel 250 positioned proximally of the clot material 1381. The embolic protection device 136 can be deployed/expanded as described previously with reference to block 1093 and FIG. 11C, e.g., by retracting the catheter 120 (e.g., proximally and/or over the first shaft 140) to uncover the embolic protection device 136. When expanded/deployed, the embolic protection device 136 can span all or a portion of the width/diameter of the blood vessel 780, and block or substantially block blood flow through the blood vessel 780.

At block 1294, the method 1290 can include positioning a clot treatment device within the first shaft of the embolic protection device. For example, FIG. 13D shows the deployment shaft 141 advanced through and positioned within the first shaft 140. The deployment shaft 141 can cover and/or constrain the clot treatment device 138 (FIGS. 8A and 8B). Accordingly, the clot treatment device 138 can be advanced together with the deployment shaft 141 while constrained therein. In some embodiments, at least a portion of the deployment shaft 141 can extend distally beyond the first shaft 140 and/or the clot material 1381, as shown in FIG. 13D. During insertion, the first actuation component 132 and the third actuation component 139 can be spaced apart from one another. For example, as described above with reference to FIG. 1B, the first actuation component 132 and/or the second shaft 142 can be pinned in position relative to the third actuation component 139 such that the clot treatment device 138 is constrained within the deployment shaft 141.

At block 1295, the method 1290 can include deploying the clot treatment device at least partially distal to clot material in the blood vessel. For example, FIG. 13E shows the clot treatment device 138 of FIGS. 1A-1C deployed/expanded within the blood vessel 780, with at least a portion of the clot treatment device 138 positioned distal to the clot material 781. The clot treatment device 138 can be deployed/expanded as described previously with reference to block 1092 and/or FIG. 11B, e.g., by retracting the deployment shaft 141 (e.g., proximally and/or over the second shaft 142) to uncover the clot removal element 1360 carried thereby. To withdraw the deployment shaft 141, the third actuation component 139 can be withdrawn over the second shaft 142 toward the first actuation component 132, decreasing the distance between the third actuation component 139 and the first actuation component 132, e.g., until the third actuation component 139 contacts the first actuation component 132 as shown in FIG. 13E.

At block 1296, the method 1290 can include mechanically disrupting the clot material in the blood vessel with the clot treatment device. For example, FIG. 13F shows the clot treatment device 138 (e.g., the clot removal element 1360, shown in FIG. 13E) retracted (e.g., proximally) to capture at least a portion of the clot material 1381. This can be at least generally similar to or the same as the process described with reference to block 1094 and/or FIG. 11D. For example, the clot treatment device 138 can be retracted through/against the clot material 1381 toward the embolic protection device 136 and/or at least partially into the interior 258 of the funnel 250. The embolic protection device 136 (e.g., the funnel 250) can be positioned proximal to the clot material 1381 and configured to inhibit or even prevent blood flow (in the direction shown by the arrow B) through the blood vessel 780. In some aspects of the present technology, blocking blood flow through the blood vessel 780 via the embolic protection device 136 can inhibit portions (e.g., fragments) of the clot material 1381 from migrating downstream farther into the vasculature of the patient. Additionally, the embolic protection device 136 can direct the clot removal element 1360 and the captured clot material contained within the clot removal element 1360 into the first shaft 140. Accordingly, the embolic protection device 136 is expected to capture and/or direct all, or at least a portion, of the clot material 1381 into the first shaft 140. In some embodiments, after the clot material 1381 is sufficiently disrupted, block 1296 can include removing the clot treatment device from the patient, e.g., by retracting (e.g., proximally) one or both of the deployment shaft 141 and the second shaft 142 into and/or through the first shaft 140 and/or the catheter 120. In these and/or other embodiments, the clot treatment device can be reintroduced one or more times as needed to remove additional clot material in a manner at least generally similar or identical to blocks 1294 and/or 1295.

In some embodiments, at block 1297, the method 1290 includes aspirating clot material from the blood vessel through a lumen of the clot treatment system 100. The aspirated clot material can include all or a portion of the clot material remaining in the blood vessel after the mechanical disruption in block 1296. For example, FIG. 13G shows remaining clot material 1384 being aspirated from the blood vessel 780 via the first shaft 140. The embolic protection device 136 can remain deployed the aspiration, e.g., to direct the remaining clot material 1384 into the first shaft 140 and/or inhibit or even prevent the remaining clot material 1384 from embolizing. In these and/or other embodiments, another shaft of the system 100 (e.g., the catheter 120) can be used to aspirate the remaining clot material 1384 and/or other material from the blood vessel 780. In some embodiments, blocks 1296 and 1297 can be repeated until all, or at least a sufficient quantity, of the material in the vasculature has been removed, via one or both of mechanical disruption (e.g., block 1296) and aspiration (e.g., block 1297). The sufficient quantity can be at least 50%, 60%, 70%, 80%, 90%, 95%, 99%, etc., of the clot material at a given location in the patient. Although block 1297 is shown as occurring after block 1296 in FIG. 12, in other embodiments block 1297 can occur before block 1296 and/or at a same time as block 1296. After the clot material has been sufficiently removed, the method 1290 can include removing the clot treatment system 100 from the patient (block 1298).

Several aspects of the present technology are set forth in the following examples:
1. A system for removing clot material from a blood vessel of patient, the system comprising:
   a delivery catheter;
   an intermediate catheter configured to extend through the delivery catheter;
   an embolic protection device coupled to a distal portion of the intermediate catheter, wherein the delivery catheter and intermediate catheter are movable relative to one another to move the embolic protection device between (a) a first embolic protection device position in which the embolic protection device is constrained within the delivery catheter and (b) a second embolic protection device position in which the embolic protection device is unconstrained by the delivery catheter and configured to expand within the blood vessel;
   a deployment catheter configured to extend through the intermediate catheter;
   an elongate shaft configured to extend through the deployment catheter; and
   a clot treatment device coupled to the elongate shaft, wherein the deployment catheter and the intermediate catheter are movable relative to one another to move the embolic protection device between (a) a first clot treatment device position in which the clot treatment device is constrained within the deployment catheter and (b) a second clot treatment device position in which the clot treatment device is unconstrained by the deployment catheter and configured to expand within the blood vessel.
2. The system of example 1 wherein, in the second embolic protection device position, the embolic protection device is configured to expand to a diameter of the blood vessel.
3. The system of example 2 wherein the embolic protection device is impermeable to blood.
4. The system of example 2 or example 3 wherein, in the second embolic protection device position, the embolic protection device is configured to substantially prevent blood flow through the blood vessel.
5. The system of any one of examples 1-4 wherein the blood vessel is a periphery artery.
6. The system of any one of examples 1-5 wherein the embolic protection device is a funnel having a tapered shape.
7. The system of example 6 wherein the embolic protection device is impermeable to blood.
8. The system of any one of examples 1-7 wherein the embolic protection device is a balloon.
9. The system of example 8 wherein, in the second embolic protection device position, the balloon is configured to expand to a diameter of the blood vessel to substantially prevent blood flow through the blood vessel.
10. The system of any one of examples 1-9, further comprising:
   a valve assembly fixedly coupled to a proximal portion of the deployment catheter; and
   a hub fixedly coupled to a proximal portion of the elongate shaft, wherein the valve assembly is movable relative to the hub to retract the deployment catheter relative to the elongate shaft to move the clot treatment device between the first clot treatment position and the second clot treatment position.
11. The system of any one of examples 1-10, further comprising a handle having a housing and a trigger movable relative to the housing, wherein the deployment catheter is fixedly coupled to the trigger, and wherein the trigger is movable relative to housing to retract the deployment catheter relative to the elongate shaft to move the clot treatment device between the first clot treatment position and the second clot treatment position.
12. The system of example 11, further comprising a hub fixedly coupled to a proximal portion of the elongate shaft, wherein the hub is coupled to the housing.
13. The system of any one of examples 1-12, further comprising an aspiration source fluidly coupled to the delivery catheter.
14. A method of removing clot material from a blood vessel of a patient, the method comprising:
   advancing a delivery catheter through the blood vessel such that a distal portion of the delivery catheter is proximate to the clot material;
   advancing an embolic protection device through the delivery catheter, wherein the embolic protection device is coupled to a distal portion of an intermediate catheter;
   retracting the delivery catheter to allow the embolic protection device to expand within the blood vessel at least partially proximal to the clot material;
   advancing a clot treatment assembly through the intermediate catheter and at least partially through the clot material;
   retracting a deployment catheter of the clot treatment assembly relative to a clot treatment device of the clot treatment assembly to allow the clot treatment device to expand within the blood vessel at least partially distal to the clot material; and
   withdrawing the clot treatment device proximally though the clot material and into the embolic protection device.
15. The method of example 14 wherein the method further comprises substantially inhibiting blood flow through the blood vessel after expanding the embolic protection device.
16. The method of example 14 or example 15 wherein allowing the embolic protection device to expand includes allowing the embolic protection device to expand to a diameter of the blood vessel.
17. The method of any one of examples 14-16 wherein the embolic protection device is impermeable to blood.
18. The system of any one of examples 14-17 wherein the blood vessel is a periphery artery.
19. The system of any one of examples 14-18 wherein the embolic protection device is a funnel having a tapered shape.
20. The system of any one of examples 14-19 wherein the embolic protection device is a balloon, and wherein allowing the embolic protection device to expand includes inflating the balloon.
21. A method of removing clot material from a blood vessel of a patient using a clot treatment system, the method comprising:
   inserting a catheter to proximate the clot material within the blood vessel while radially constraining a clot treatment device and an embolic protection device within the catheter;
   moving the catheter proximally to deploy the clot treatment device at least partially distal to clot material within the blood vessel; and
   moving the catheter further proximally to deploy the embolic protection device at least partially proximal to the clot material.
22. The method of example 21, further comprising mechanically disrupting at least part of the clot material by retracting the clot treatment device through the clot material toward the catheter.
23. The method of example 21 or example 22, further comprising retracting the clot treatment device through the clot material and at least partially into the embolic protection device.
24. The method of any one of examples 21-23, further comprising aspirating a portion of the clot material via a lumen defined at least partially by the clot treatment system.
25. The method of example 24 wherein the lumen is a catheter lumen defined by the catheter.
26. The method of example 24 or example 25, wherein the clot treatment device is carried by a shaft extending through the catheter, and wherein the lumen is a shaft lumen defined by the shaft.
27. The method of any one of examples 24-26 wherein the embolic protection device is carried by a shaft extending through the catheter, and wherein the lumen is a shaft lumen defined by the shaft.
28. The method of any one of examples 21-27 wherein inserting the catheter includes directly contacting the clot treatment device and the embolic protection device with an inner surface of the catheter to radially-constrain the clot treatment device and the embolic protection device.
29. A system for removing clot material from a vasculature of patient, the system comprising:
   a catheter defining a lumen; and
   a mechanical thrombectomy assembly configured to be positioned at least partially within the lumen, the mechanical thrombectomy assembly including—
      a first shaft;
      a second shaft configured to be positioned at least partially within the first shaft;
      an embolic protection device coupled to the first shaft; and
      a clot treatment device coupled to the second shaft and positioned distal of the embolic protection device;
   wherein, in response to proximal movement of the catheter relative to the mechanical thrombectomy assembly, the clot treatment device and the embolic protection device are configured to transition between an undeployed configuration and a deployed configuration.
30. The system of example 29 wherein the embolic protection device includes a funnel defining a distal-facing opening.
31. The system of example 29 wherein the embolic protection device includes a balloon.
32. The system of any one of examples 29-31 wherein at least part of the second shaft contacts an inner surface of the first shaft.
33. The system of any one of examples 29-32 wherein the clot treatment device and the embolic protection device are configured to transition between the undeployed configuration and the deployed configuration sequentially during the proximal movement of the catheter.
34. A method of removing clot material from a blood vessel of a patient using a clot treatment system, the method comprising:
inserting a catheter to proximate the clot material within the blood vessel while radially constraining a clot treatment device and an embolic protection device within the catheter;
moving an intermediate deployment shaft of the clot treatment system proximally to deploy the clot treatment device at least partially distal to the clot material within the blood vessel, wherein the clot treatment device is carried by a first shaft of the clot treatment system, and wherein the intermediate deployment shaft is positioned radially outward of the first shaft; and
moving the catheter proximally to deploy the embolic protection device at least partially proximal to the clot material, wherein the embolic protection device is carried by a second shaft of the clot treatment system, and wherein the second shaft is configured to receive the intermediate deployment shaft.
35. The method of example 34, further comprising mechanically disrupting at least part of the clot material by retracting the clot treatment device through the clot material toward the catheter.
36. The method of example 35, further comprising retracting the clot treatment device through the clot material and at least partially into the embolic protection device.
37. The method of example 36, further comprising aspirating at least part of the clot material via a lumen defined at least partially by the clot treatment system.
38. The method of example 37 wherein the lumen is a catheter lumen defined by the catheter.
39. The method of example 37 wherein the lumen is deployment lumen defined by the intermediate deployment shaft.
40. The method of any one of examples 34-39 wherein inserting the clot treatment system includes directly contacting the embolic protection device with an inner surface of the catheter to radially-constrain the embolic protection device while advancing the catheter and the embolic protection device together.
41. The method of any one of examples 34-40 wherein inserting the clot treatment system includes advancing the catheter with (a) the embolic protection device and the clot treatment device positioned therein and (b) the clot treatment device positioned distal to the embolic protection device.
42. The method of any one of examples 34-41, further comprising positioning the embolic protection device within the catheter prior to deploying the embolic protection device.
43. The method of example 42, further comprising positioning the clot treatment device within the second shaft prior to deploying the embolic protection device.
44. The method of example 43 wherein positioning the embolic protection device is within the catheter and deploying the embolic protection device from the catheter occur prior to positioning the clot treatment device within the second shaft.
45. The method of example 44 wherein deploying the clot treatment device includes deploying the clot treatment device distally of the deployed embolic protection device.
46. A system for removing clot material from a blood vessel of patient, the system comprising:
   a delivery catheter;
   an intermediate catheter configured to extend through the delivery catheter;
   a funnel coupled to a distal portion of the intermediate catheter, wherein the delivery catheter and intermediate catheter are movable relative to one another to move the funnel between (a) a first funnel position in which the funnel is constrained within the delivery catheter and (b) a second funnel position in which the funnel is unconstrained by the delivery catheter and configured to expand within the blood vessel;
   a deployment catheter configured to extend through the intermediate catheter;
   an elongate shaft configured to extend through the deployment catheter; and
   a clot treatment device coupled to the elongate shaft, wherein the deployment catheter and the intermediate catheter are movable relative to one another to move the embolic protection device between (a) a first clot treatment device position in which the clot treatment device is constrained within the deployment catheter and (b) a second clot treatment device position in which the clot treatment device is unconstrained by the deployment catheter and configured to expand within the blood vessel.
47. The system of example 46 wherein, in the second funnel position, the funnel is configured to expand to a diameter of the blood vessel.
48. The system of example 47 wherein the funnel is impermeable to blood.
49. The system of example 47 or example 48 wherein, in the second funnel position, the funnel is configured to substantially prevent blood flow through the blood vessel.
50. The system of any one of examples 46-49 wherein the blood vessel is a periphery artery.
51. The system of any one of examples 46-50 wherein the funnel has a tapered shape.
52. The system of example 51 wherein the funnel is impermeable to blood.
53. The system of any one of examples 46-52, further comprising:
   a valve assembly fixedly coupled to a proximal portion of the deployment catheter; and
   a hub fixedly coupled to a proximal portion of the elongate shaft, wherein the valve assembly is movable relative to the hub to retract the deployment catheter relative to the elongate shaft to move the clot treatment device between the first clot treatment position and the second clot treatment position.
54. The system of any one of examples 46-53, further comprising a handle having a housing and a trigger movable relative to the housing, wherein the deployment catheter is fixedly coupled to the trigger, and wherein the trigger is movable relative to housing to retract the deployment catheter relative to the elongate shaft to move the clot treatment device between the first clot treatment position and the second clot treatment position.
55. The system of example 54, further comprising a hub fixedly coupled to a proximal portion of the elongate shaft, wherein the hub is coupled to the housing.
56. The system of any one of examples 46-55, further comprising an aspiration source fluidly coupled to the delivery catheter.
57. A method of removing clot material from a blood vessel of a patient, the method comprising:
   advancing a delivery catheter through the blood vessel such that a distal portion of the delivery catheter is proximate to the clot material;
   advancing a funnel through the delivery catheter, wherein the funnel is coupled to a distal portion of an intermediate catheter;
   retracting the delivery catheter to allow the funnel to expand within the blood vessel at least partially proximal to the clot material;
   advancing a clot treatment assembly through the intermediate catheter and at least partially through the clot material;
   retracting a deployment catheter of the clot treatment assembly relative to a clot treatment device of the clot treatment assembly to allow the clot treatment device to expand within the blood vessel at least partially distal to the clot material; and
   withdrawing the clot treatment device proximally though the clot material and into the funnel.
58. The method of example 57 wherein the method further comprises substantially inhibiting blood flow through the blood vessel with the funnel after expanding the funnel.
59. The method of example 57 or example 58 wherein allowing the funnel to expand includes allowing the funnel to expand to a diameter of the blood vessel.
60. The method of any one of examples 57-59 wherein the funnel is impermeable to blood.
61. The system of any one of examples 57-60 wherein the blood vessel is a periphery artery.
62. The system of any one of examples 57-61 wherein the funnel is a funnel having a tapered shape.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/we claim:

1. A system for removing clot material from a blood vessel of patient, the system comprising:
a delivery catheter;
an intermediate catheter configured to extend through the delivery catheter;
an embolic protection device coupled to a distal portion of the intermediate catheter, wherein the delivery catheter and intermediate catheter are movable relative to one another to move the embolic protection device between (a) a first embolic protection device position in which the embolic protection device is constrained within the delivery catheter and (b) a second embolic protection device position in which the embolic protection device is unconstrained by the delivery catheter and configured to expand within the blood vessel;
a deployment catheter configured to extend through the intermediate catheter;
an elongate shaft configured to extend through the deployment catheter; and
a clot treatment device coupled to the elongate shaft, wherein the deployment catheter and the intermediate catheter are movable relative to one another to move the embolic protection device between (a) a first clot treatment device position in which the clot treatment device is constrained within the deployment catheter and (b) a second clot treatment device position in which the clot treatment device is unconstrained by the deployment catheter and configured to expand within the blood vessel.

2. The system of claim 1 wherein, in the second embolic protection device position, the embolic protection device is configured to expand to a diameter of the blood vessel.

3. The system of claim 2 wherein the embolic protection device is impermeable to blood.

4. The system of claim 2 wherein, in the second embolic protection device position, the embolic protection device is configured to substantially prevent blood flow through the blood vessel.

5. The system of claim 1 wherein the blood vessel is a periphery artery.

6. The system of claim 1 wherein the embolic protection device is a funnel having a tapered shape.

7. The system of claim 6 wherein the embolic protection device is impermeable to blood.

8. The system of claim 1 wherein the embolic protection device is a balloon.

9. The system of claim 8 wherein, in the second embolic protection device position, the balloon is configured to expand to a diameter of the blood vessel to substantially prevent blood flow through the blood vessel.

10. The system of claim 1, further comprising:
a valve assembly fixedly coupled to a proximal portion of the deployment catheter; and
a hub fixedly coupled to a proximal portion of the elongate shaft, wherein the valve assembly is movable relative to the hub to retract the deployment catheter relative to the elongate shaft to move the clot treatment device between the first clot treatment position and the second clot treatment position.

11. The system of claim 1, further comprising a handle having a housing and a trigger movable relative to the housing, wherein the deployment catheter is fixedly coupled to the trigger, and wherein the trigger is movable relative to housing to retract the deployment catheter relative to the elongate shaft to move the clot treatment device between the first clot treatment position and the second clot treatment position.

12. The system of claim 11, further comprising a hub fixedly coupled to a proximal portion of the elongate shaft, wherein the hub is coupled to the housing.

13. The system of claim 1, further comprising an aspiration source fluidly coupled to the delivery catheter.

14. A method of removing clot material from a blood vessel of a patient, the method comprising:
advancing a delivery catheter through the blood vessel such that a distal portion of the delivery catheter is proximate to the clot material;
advancing an embolic protection device through the delivery catheter, wherein the embolic protection device is coupled to a distal portion of an intermediate catheter;
retracting the delivery catheter to allow the embolic protection device to expand within the blood vessel at least partially proximal to the clot material;
advancing a clot treatment assembly through the intermediate catheter and at least partially through the clot material;
retracting a deployment catheter of the clot treatment assembly relative to a clot treatment device of the clot treatment assembly to allow the clot treatment device to expand within the blood vessel at least partially distal to the clot material; and
withdrawing the clot treatment device proximally though the clot material and into the embolic protection device.

15. The method of claim 14 wherein the method further comprises substantially inhibiting blood flow through the blood vessel after expanding the embolic protection device.

16. The method of claim 14 wherein allowing the embolic protection device to expand includes allowing the embolic protection device to expand to a diameter of the blood vessel.

17. The method of claim 14 wherein the embolic protection device is impermeable to blood.

18. The method of claim 14 wherein the blood vessel is a periphery artery.

19. The method of claim 14 wherein the embolic protection device is a funnel having a tapered shape.

20. The method of claim 14 wherein the embolic protection device is a balloon, and wherein allowing the embolic protection device to expand includes inflating the balloon.

21. A method of removing clot material from a blood vessel of a patient using a clot treatment system, the method comprising:
inserting a catheter to proximate the clot material within the blood vessel while radially constraining a clot treatment device and an embolic protection device within the catheter;
moving the catheter proximally to deploy the clot treatment device at least partially distal to clot material within the blood vessel; and
moving the catheter further proximally to deploy the embolic protection device at least partially proximal to the clot material.

22. The method of claim 21, further comprising mechanically disrupting at least part of the clot material by retracting the clot treatment device through the clot material toward the catheter.

23. The method of claim 21, further comprising retracting the clot treatment device through the clot material and at least partially into the embolic protection device.

24. The method of claim 21, further comprising aspirating a portion of the clot material via a lumen defined at least partially by the clot treatment system.

25. The method of claim 24 wherein the lumen is a catheter lumen defined by the catheter.

26. The method of claim 24, wherein the clot treatment device is carried by a shaft extending through the catheter, and wherein the lumen is a shaft lumen defined by the shaft.

27. The method of claim 24 wherein the embolic protection device is carried by a shaft extending through the catheter, and wherein the lumen is a shaft lumen defined by the shaft.

28. The method of claim 21 wherein inserting the catheter includes directly contacting the clot treatment device and the embolic protection device with an inner surface of the catheter to radially-constrain the clot treatment device and the embolic protection device.

29. A system for removing clot material from a vasculature of patient, the system comprising:
a catheter defining a lumen; and
a mechanical thrombectomy assembly configured to be positioned at least partially within the lumen, the mechanical thrombectomy assembly including
a first shaft;
a second shaft configured to be positioned at least partially within the first shaft;
an embolic protection device coupled to the first shaft; and
a clot treatment device coupled to the second shaft and positioned distal of the embolic protection device;
wherein, in response to proximal movement of the catheter relative to the mechanical thrombectomy assembly, the clot treatment device and the embolic protection device are configured to transition between an undeployed configuration and a deployed configuration.

30. The system of claim 29 wherein the embolic protection device includes a funnel defining a distal-facing opening.

31. The system of claim 29 wherein the embolic protection device includes a balloon.

32. The system of claim 29 wherein at least part of the second shaft contacts an inner surface of the first shaft.

33. The system of claim 29 wherein the clot treatment device and the embolic protection device are configured to transition between the undeployed configuration and the deployed configuration sequentially during the proximal movement of the catheter.

34. A method of removing clot material from a blood vessel of a patient using a clot treatment system, the method comprising:
inserting a catheter to proximate the clot material within the blood vessel while radially constraining a clot treatment device and an embolic protection device within the catheter;
moving an intermediate deployment shaft of the clot treatment system proximally to deploy the clot treatment device at least partially distal to the clot material within the blood vessel, wherein the clot treatment device is carried by a first shaft of the clot treatment system, and wherein the intermediate deployment shaft is positioned radially outward of the first shaft; and
moving the catheter proximally to deploy the embolic protection device at least partially proximal to the clot material, wherein the embolic protection device is carried by a second shaft of the clot treatment system, and wherein the second shaft is configured to receive the intermediate deployment shaft.

35. The method of claim 34, further comprising mechanically disrupting at least part of the clot material by retracting the clot treatment device through the clot material toward the catheter.

36. The method of claim 35, further comprising retracting the clot treatment device through the clot material and at least partially into the embolic protection device.

37. The method of claim 36, further comprising aspirating at least part of the clot material via a lumen defined at least partially by the clot treatment system.

38. The method of claim 37 wherein the lumen is a catheter lumen defined by the catheter.

39. The method of claim 37 wherein the lumen is deployment lumen defined by the intermediate deployment shaft.

40. The method of claim 34 wherein inserting the clot treatment system includes directly contacting the embolic protection device with an inner surface of the catheter to radially-constrain the embolic protection device while advancing the catheter and the embolic protection device together.

41. The method of claim 34 wherein inserting the clot treatment system includes advancing the catheter with (a) the embolic protection device and the clot treatment device positioned therein and (b) the clot treatment device positioned distal to the embolic protection device.

42. The method of claim 34, further comprising positioning the embolic protection device within the catheter prior to deploying the embolic protection device.

43. The method of claim 42, further comprising positioning the clot treatment device within the second shaft prior to deploying the embolic protection device.

44. The method of claim 43 wherein positioning the embolic protection device is within the catheter and deploying the embolic protection device from the catheter occur prior to positioning the clot treatment device within the second shaft.

45. The method of claim 44 wherein deploying the clot treatment device includes deploying the clot treatment device distally of the deployed embolic protection device.
